(19) United States
(12) Patent Application Publication
(10) Pub. No.: US 2004/0109871 A1
(43) Pub. Date: Jun. 10, 2004

(54) M cell directed vaccines

(76) Inventor: David W. Pascual, Bozeman, MT (US)

Correspondence Address: MORGAN LEWIS & BOCKIUS LLP, 1111 PENNSYLVANIA AVENUE NW, WASHINGTON, DC, 20004, US

(21) Appl. No.: 10/660,787

(22) Filed: Sep. 12, 2003

Related U.S. Application Data
(63) Continuation-in-part of application No. PCT/US02/07254, filed on Mar. 12, 2002.
(63) Continuation-in-part of application No. 10/169,492, filed on Oct. 21, 2002.
Application No. 10/169,492 is a 371 of international application No. PCT/US01/00426, filed on Jan. 8, 2001.
(60) Provisional application No. 60/274,639, filed on Mar. 12, 2001.
(60) Provisional application No. 60/174,786, filed on Jan. 6, 2000.

(51) Int. Cl. A61K 48/00; A61K 39/00

(52) U.S. Cl. 424/185.1; 514/44

(57) ABSTRACT

This invention provides a vaccine that can direct gene transfer and the transfer of other immunogens to follicle associated epithelium or M cells to induce mucosal immunity using M cell ligands for receptor-mediated endocytosis. Also provided are polynucleotides sequences encoding M cell ligand-polybasic component fusion proteins, host cells, and methods of producing such proteins recombinantly and chemically. Further, methods are described for immunizing animal and human subjects against bacterial, viral, parasitic, fungal infectious agents or cancer and methods for assaying mucosal immunity using this vaccine.

### RELATED APPLICATIONS

This application claims priority to Application Serial No. 60/274,639, and is related to PCT Application, PCT/US01/00426, filed Jan. 8, 2001, which claims priority to U.S. Provisional Application No. 60/174,786, filed Jan. 6, 2000.

### TECHNICAL FIELD

The present invention is in the general field of vaccine development. The present invention provides methods and compositions useful for, among other purposes, the identification, diagnosis, prevention and treatment of bacterial, viral, parasitic, fungal infectious agents or cancer for human, livestock, and wildlife. More specifically, the present invention provides DNA and other vaccines directed to follicle-associated epithelium. Even more specifically, the invention is directed to polycation conjugated M cell ligand (e.g., enteric adhesins)—DNA complex vaccine compositions and diagnostic and therapeutic uses thereof.

### BACKGROUND OF THE INVENTION

Aspects of this invention are discussed in Wu et al., Gene Therapy (2000) 7(1):61-69, and in Wu et al., Proc. Natl. Acad. Sci. USA (2001) 98:9318-23, each of which is herein incorporated by reference in its entirety.

All publications and patent applications mentioned or identified in this specification are incorporated by reference to the same extent as if each individual publication or patent application was specifically and individually indicated to be incorporated by reference.

Recent studies have shown the utility of DNA vaccination for inducing protective immunity in experimental animals exposed to influenza (Fynan et al., Proc Natl Acad Sci USA (1993) 90:11478-11482 and Robinson et al., Vaccine (1993) 11:957-960), herpes simplex virus (HSV)(Gillichan et al., J Inf Dis (1998) 177:1155-1161), HIV-1 (Boyer), rotavirus (Herrmann et al., J Infec Dis (1996) 174(Suppl.1):S93-S97 and Chen et al., J Virol (1998) 72:5757-5761), and Borrelia burgdorgeri infections (Simon et al., J Immunol (1996) 26:2831-2840). DNA immunization has a number of attractive features including ease of preparation for encoding desired protective immunogens, co-expression of immunogens, co-expression of adjuvant (e.g., cytokines), no requirement for large-scale protein purifications, and ease of delivery. However, conventional DNA vaccine technology immunizes the host at peripheral sites, e.g., intradermal or intramuscular sites. While these methods can elicit systemic cell-mediated and antibody-dependent responses, most infectious agents infect via a mucosal surface, and such DNA immunizations at peripheral sites do not result in optimal mucosal immunity (i.e., both antibody, particularly IgA, and cellular (cytotoxic T lymphocyte (CTL) immunity induction).

This lack of mucosal immunity induction has prompted attempts to deliver DNA vaccines to mucosal surfaces. For example, successful induction of mucosal immunity has been accomplished using DNA vaccines by intraoral jet delivery (Chen et al., Vaccine (1999) 17(23-24):3171-3176); co-administration of a DNA vaccine with a polymer by intranasal injection (Hamajima et al., Clinical Immunol Immunopathol (1998) 88(2):205-210); co-administration of a DNA vaccine with IL-12 (Okada et al., J Immnunol (1997) 159(7):3638-3647); intravaginal administration of DNA vaccines (Wang et al., Vaccine (1997) 15(8):821-825); oral delivery of micro-encapsulated DNA vaccines (Jones et al., Dev Biol Stand (1998) 92:149-155); and parenteral and mucosal injection of DNA vaccines (Shroff et al., Vaccine (1999) 18(3-4):222-230). However, these methods generally lack mucosal surface selectivity. Nevertheless, they illustrate the desire to observe mucosal immunity as the end-point in determining the efficacy of these vaccines.

Transepithelial transport of antigens and pathogens is the first step in the induction of a mucosal immune response. Mucosal inductive tissues are sites in the small intestine or in the nasal passages where vaccine antigens are taken to be processed and presented to mucosal lymphocytes for the development of mucosal immunity (Frey et al., Behring Inst Mitt (1997) 98:376-389). In the intestine, the delivery of antigen across the epithelial barrier to the underlying lymphoid tissue is accomplished by M cells, a specialized epithelial cell type that occurs only in the lymphoid follicle-associated epithelium (Frey et al., 1997). Further, such follicle-associated epithelium is found in the nasal lymphoid tissues (believed to be sites of induction of mucosal immune responses to airborne antigens; Giannasca et al., Infect Immun (1997) 65(10):4288-4289). Selective and efficient transport of antigen by M cells is considered an essential requirement for effective mucosal vaccines. Thus, targeting of M cells by taking advantage of their capacity to endocytose particles, including those particles comprising gene transfer vehicles and DNA vaccines, has generated great interest as selective transfer of genes across the follicle-associated epithelium would be advantageous from both investigational and therapeutic standpoints.

Although viruses can be efficient gene transfer vehicles, progress has been made toward developing non-viral delivery systems. Coupling of a specific ligand to vaccines or drugs can be a powerful aid to route compounds to a certain target population. One of the most promising means is by exploiting receptor-mediated endocytosis pathways using selective ligands. In this method, DNA-ligand complexes are internalized by targeted cells when the ligand binds to its respective cell-surface receptor. Such receptor-mediated gene transfer has been accomplished using a variety of receptors by conjugating DNA to their cognate ligands such as asialo-orosomucoid (Wu et al., J Biol Chem (1989) 264:16985-16987 and Wu et al., J Biol Chem (1994) 269:11542-11546), transferrin (Lozier et al., Human Gene Ther (1994) 5:313-322; Wagner et al., Proc Natl Acad Sci USA (1990) 87:3410-3414; and Wagner et al., Proc Natl Acad Sci USA (1992) 89:6099-6103), lectins (Batra et al., Gene Ther (1994) 1:255-260), folate (Leamon et al., Biochem J (1993)291:855-860), lung surfactant protein (Ross et al., Human Gene Ther (1995) 6:31-40), insulin (Sobolev et al., J Biol Chem (1998) 273:7928-7933) and would include receptor specific monoclonal antibodies (Chen et al., FEBS Lett (1994) 338:167-169 and Kang et al., J Pharmacol Exp Therapeut (1994) 269:344-350).

Receptor-mediated gene transfer has some advantages over the other methods of in vivo gene transfer. Compared to attenuated viral vectors, it shares tissue-specificity, but receptor-mediated gene transfer minimizes the use of viral gene elements, obviating the concerns regarding genomic integration. Further, it lessens concerns with the proinflammatory properties often associated with viral vectors (Simon et al., Human Gene Ther (1993) 4:771-780; Yang et al., J Virol (1996) 70:7209-7212; and van Ginkel et al., J Immunol (1997) 159:685-693). The DNA-ligand complex is believed to be internalized by receptor-dependent endocytosis rendering transfection to be minimally toxic. The conjugate carrier complex can be designed for cell-specific targeting by selecting the appropriate receptor ligand. For example, efficient transfer of DNA to the intestinal epithelial cells by transferrin-polylysine conjugates and M cell lectins have been used to successfully transfect gastrointestinal cells in vitro (Batra et al., Cancer Gene Ther (1994) 1(3):185-192 and Curiel et al., Am J Respir Cell Mol Biol (1992) 6(3):247-252). However, as transferrin receptors are not restricted to M cells or follicle associated epithelium and as M cell lectins can potentially bind to any α-linked galactose (Giannasca et al., 1997), the use of these systems in vivo is limited.

The surface properties of many enteric pathogens are important in the establishment of the pathogen in the host. For example, enteropathic Escherichia coli (EPEC) induce characteristic attaching and effacing (A/E) lesions on epithelial cells of Peyer's patches (Hartland et al., Mol Microbiol (1999) 32(1):151-158). This event is mediated, in part, by binding of the bacterial outer membrane protein, intimin, to a second EPEC protein, Tir (translocated intimin receptor), which is exported by the bacteria and integrated into the host cell plasma membrane. Both of these protein have been shown to bind to host cells in vitro (Hartland et al., 1999 and DeVinney et al., Cell Mol Life Sci (1999) 55(6-7):961-976).

Reovirus is an enteric pathogen and infects the host following attachment to intestinal Peyer's patch M cells (Lee et al., Virology (1981) 108:156-63 and Mah et al., J Virol (1990) 179:95-103). Thus, as with other enteric pathogens, reovirus exploits M cells as a means to gain entry into the host. Mediating reovirus attachment is the adhesin, σ1, which is expressed as a viral coat protein (Lee et al., 1981). The protein σ1 is a 45 kilodalton protein that polymerizes via its N-terminus (Mah et al., 1990) to form a tetramer when isolated from reovirus-infected cells or purified as a recombinant protein from E. coli (Bassel-Assel-Duby et al., J Virol (1987) 61:1834-1841). In vitro analysis has demonstrated that neutral liposomes comprising σ1 protein can be taken up by rat Peyer's patches (Rubas et al., J Microencapsul (1990) 7(3):385-395). Thus, enteric pathogen adhesins make more effective targeting ligands than either transferrin or M cell lectins (Batra et al., 1994, Curiel et al., 1992 and Giannasca et al., 1997).

This invention exploits receptor mediated endocytosis as a means of delivering antigens to mucosal lymphoid tissue and introducing DNA into cells using M cell ligands for specific targeting of DNA to follicle associated epithelium of nasal or gastrointestinal origin. We have discovered that, by chemically coupling M cell ligands to a polymeric chain of basic amino acids (e.g., polylysine) and allowing that construct to associate or complex with DNA, the DNA (or RNA or other nucleic acid) can be delivered to appropriate tissue types to obtain enhanced in vivo mucosal IgA antibody and T cell responses against an encoded antigen.

Further, to demonstrate the efficacy of the present vaccine design, we have applied this concept to reporter gene products, β-galactosidase and luciferase, as well as vaccine antigens derived from human immunodeficiency virus (HIV) and Brucella in vivo. Using these systems, enhanced mucosal IgA antibody responses can be demonstrated between animals vaccinated with DNA only (that is, DNA not included in our formulation) and those vaccinated with conjugated DNA complexes.

### SUMMARY OF THE INVENTION

The present invention is based, in part, on the observation that a DNA vaccine protected from the mucosal environment can be effectively used to vaccinate a host by targeting the mucosa. Data described herein shows that appropriately formulated DNA constructs show improved mucosal IgA antibody responses when compared to DNA applied directly to a mucosal surface. The present invention is further based on the induced anti-vaccine antibody and cellular immune responses produced by vaccinated mice, cattle, and bison. Based on these observations, the present invention provides compositions and methods for use in a variety of animals, particularly humans, livestock, and wildlife.

It is therefore an object of this invention to provide a method for inducing mucosal immunity using receptor mediated endocytosis pathways to deliver nucleotide, particularly DNA, vaccines, as well as other immunogens, to specific cells of the follicle associated epithelium, preferably M-cells, for example, of nasal and gastrointestinal origin. It is also an object of this invention to provide DNA and other vaccine compositions comprising a polypeptide (or other complexing agent) linked electrostatically to (or otherwise associated or complexed with) a DNA structural sequence or gene or other immunogen. Particularly contemplated are polypeptide-DNA complexes, in which the polypeptide is comprised of a polymeric chain of basic amino acid residues and an M cell specific ligand.

The DNA structural sequence preferably encodes an immunogenic antigen from an infectious agent, but also may encode other immunogens, such as a tumor specific antigen, against which the induction of an immune response is desired, but also including antigens against which a host might be tolerized. The present invention provides the ability to produce a previously unknown protein—and to elicit an immune response against such proteins—using the cloned nucleic acid molecules derived, for example, from any given infectious agent be it bacterial, fungal, viral, protozoan, parasitic or protective molecule against cancer.

Consistent with the foregoing, a preferred embodiment of the present invention includes an M cell specific ligand, a nucleic acid sequence encoding an immunogen, and a nucleic acid binding moiety. Preferably, the nucleic acid will be DNA although RNA vaccines are contemplated. In such vaccines, the binding moiety preferably is a polypeptide, however, other binding and complexing agents may be utilized so long as they stabilize or protect the nucleic acid and protein components of the vaccine from degradation and facilitate their delivery, by various routes of administration, to the target mucosal tissues. Thus, for example, a polypeptide binding moiety preferably comprises a polymeric chain of basic amino acid residues and a contemplated polymeric chain would comprise polylysine.

Another embodiment of the present invention includes an M cell specific ligand conjugated or complexed to an immunogen via an appropriate linker. Immunogens in this instance would include a variety of macromolecules such as peptides, proteins, lipoproteins, lipids, glycoproteins, polysaccharides, carbohydrates, some nucleic acids, and certain of the teichoic acids, or any other molecule or gene from a pathogen or tumor cell that could be used to generate a protective immune response. Such immunogens may be conjugated or complexed with the M cell specific ligand using any means known in the art. For instance, immunogens may be conjugated to an M cell specific ligand using an appropriate crosslinker. Cross-linking may be performed with either homo- or heterobifunctional agents, i.e., SPDP, DSS, SIAB. Alternatively, immunogens may be complexed with an M cell specific ligand using an appropriate complexing agent. Complexes may be formed between a 6× His tag on one molecule and a nitrilotriacetic acid-metal ion complex on the other molecule. Methods for cross-linking are disclosed in PCT/DK00/0053 1 (WO 01/22995) which is herein incorporated by reference. Alternatively, conjugates and complexes can comprise the following scenarios: polypeptides with attached immunogens may be conjugated to M cell specific ligands; liposomes can replace the polypeptide, wherein the M cell specific ligand may be conjugated to a liposome containing the immunogens, or conjugated to a liposome with one or several copies of an immunogen or different immunogens attached/displayed to its surface; and peptide and protein immunogens may be expressed as fusion proteins operably linked to the M cell specific ligand.

In general, the M cell specific ligand is selected from the group consisting of the protein σ1 of a reovirus, or is (or is derived from) an adhesin of Salmonella or a polio virus. M cell tropic fragments of the foregoing also are contemplated. In a preferred embodiment of the invention, a polypeptide binding moiety would further comprise an M cell specific ligand and may be expressed as a fusion protein.

DNA encoding protective epitopes useful for the development of M cell targeted vaccines may be obtained from the following sources. Oligonucleotide (gene) sequences encoding an epitope as described in the scientific literature or derived from genomic data that contains sequence data for microbial virulence factors may be used as the source. (Weinstock, G. M., Genomics and Bacterial Pathogenesis, Emerg. Infect. Dis., (2000) 6(5)). Another source of DNA encoding protective epitopes may be screening of epitope libraries, such as phage display libraries, as described herein.

Particularly contemplated are nucleotide and other vaccines in which the immunogen to be delivered to the target mucosal tissue is an immunogen expressed by an infectious agent such as a microorganism or is a tumor specific antigen. Preferred immunogens are derived from or, like an expressed toxin, are associated with a bacterium, protozoan, parasite, virus, fungus, prion, tuberculobacillus, leprosy bacillus, malaria parasite, diphtheria bacillus, tetanus bacillus, Leishmania, Salmonella, Schistosoma, measles virus, mumps virus, herpes virus, HIV, cancer and influenza virus.

Plasmid vectors in which DNA sequences encoding such immunogens operably linked to transcription regulatory elements are a preferred embodiment of the present invention. Moreover, the vaccines of the present invention are preferably formulated with a pharmaceutically acceptable excipient or an adjuvant such as an immunomodulator. Examples of contemplated immunomodulators include cytokines, lymphokines, interleukins, interferons and growth factors. Preferably, these vaccines induce a protective immune response in a host vaccinated against the immunogen. In other embodiments of the invention, contemplated vaccines will tolerize a host vaccinated against appropriate immunogens.

Vaccines formulated in unit dosage form, and vaccines packaged with instructions for the use of the vaccine to induce an immune response against said immunogen or disease with which said immunogen is associated are preferred. Therapeutic as well as prophylactic vaccines also are contemplated. Moreover, preferred vaccines are formulated for administration through a route selected from the group consisting of oral, nasal, vaginal, rectal and urethral routes of administration.

Another preferred embodiment of the present invention provides a method for immunizing a host against an immunogen by administering the nucleotide vaccines or other vaccines as described above. In addition, other embodiments of the invention provide a method for assaying for mucosal immunity comprising the steps of administering the vaccine to an animal which is free of infection of the infectious agent whose antigen is to be tested; isolating cells from the animal; and co-incubating said isolated cells with heterologous antigen expressing cells. In this assay, cells expressing the antigen or vaccine will be lysed. This serves as one indication that mucosal immunity was induced in the vaccinated animal. The foregoing assay method is performed using CTLs isolated from lymphoid tissue from the vaccinated animal. For example, CTLs may be isolated from Peyer's patches cells, lymph nodes, NALT, adenoids, spleens and other organized lymphoid tissue, as well as from non lymphoid tissue such as nasal passages, intestinal lamina propia, lungs, liver and vaginal epithelium.

An additional step of evaluating the animal's cytokine profile also is contemplated. Evaluation of cytokine responses is a means of measuring mucosal immunity and can give an indication of which types of cells participate in an immune response. For example, T cells can be either CTLs (CD8⁺) or helper cells (CD4⁺) that assist B cells in making antibodies. CD4⁺ Th cells may be subdivided further into at least two functionally distinct subsets, Th1 and Th2, based on the unique profiles of cytokines they produce and the major regulatory functions they play in the host's immune responses. For example, Th1 cells secrete IL-2, IFN-γ, and TNF-β, and function in cell-mediated immunity for protection against intracellular pathogens such as Listeria monocytogenes, Mycobacterium species, and Salmonella species. Th1 cells may also provide B cell help. For example, Th1 cell-derived IFN-γ favors the development of IgG2a responses in mice. Th1 cell activity is promoted by IL-12 and and IL-18. Th2 cells preferentially secrete IL-4, IL-5, IL-6, IL-10, and IL-13, and provide effective help for B cell responses, most notably for IgG1, IgE, and IgA. Promotion of Th2 cells occurs by suppression of IL-12 by Il-4 and TGF-β. Other studies have also shown that T cells and certain cytokines (e.g., IL-5 and IL-6) are of particular importance for the induction of committed surface (s)IgA⁺ B cells to differentiate into IgA-producing cells.

A related embodiment of the present invention provides an isolated nucleic acid encoding a fusion protein comprising a nucleic acid binding moiety and an M cell specific ligand. In such nucleic acids, the binding moiety encodes a polymeric chain of basic amino acid residues such as polylysine. Associated vectors comprising these nucleic acids, such as expression vectors, are expressly contemplated. Moreover, the polypeptide expression products of such vectors also may be used as immunogens in vaccines. Contemplated nucleic acids would be in an operable linkage, and would include both sense and antisense orientations relative to transcriptional elements comprising the vector. Host cells comprising or transformed with such vectors are also contemplated.

Another embodiment of the invention includes methods of expressing fusion proteins from such cells. Particularly contemplated are isolated polypeptides comprising a nucleic acid binding moiety and an M cell specific ligand. Optionally, the immunogen also may be encoded by such fusion proteins, with or without the presence of a binding moiety or interim protein sequence. It is also contemplated that antibodies may be generated that bind selectively or preferentially to such polypeptides, as opposed to the immunogen or to the M cell specific ligand or nucleic acid binding moiety themselves.

Yet another embodiment of the present invention relates to various kits for assay and other test purposes that include an M cell specific ligand and a nucleic acid binding moiety as well as the other constructs and components described above. Preferred kits will be further packaged with instructions for the use of the vaccine to induce an immune response against the immunogen or against the disease with which the immunogen is associated, for instance setting forth preferred dosage schemes and formulations as disclosed herein.

Other objects, features and advantages of the present invention will become apparent from the following detailed description. It should be understood, however, that the detailed description and specific examples, while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1 shows the cell binding capacity of recombinant protein σ1 and recombinant protein σ1-PL conjugates. Recombinant protein σ1 binds to (a) mouse L cells, (b) Caco-2 cells and (c) RFL-6 cells, as well as (d) a polylysine (PL) conjugate to mouse L cells.

FIG. 2 shows that our recombinant reovirus protein σ1 can bind murine nasal M cells.

FIG. 3 shows sustained mucosal IgA responses against the reporter gene product, luciferase.

FIG. 4 shows induced cytolytic T cell responses against the reporter gene product, β-galactosidase.

FIG. 5 shows the mucosal intestinal IgA response of mice immunized with one of three designated HIV DNA vaccine constructs presenting gp160, gp140(c) or gp 140(s).

FIGS. 6A and 6B show enhanced cytolytic activity (cell-mediated immunity) against target cells expressing HIV gp120 from biopsies from mice immunized intranasally with an M cell-formulated HIV DNA vaccine.

FIG. 7 shows that the Candida carbohydrate epitope demonstrates dose dependent inhibition of antibody-binding to mimitopes discovered through the use of phage display libraries, and also to a synthetic peptide-carrier protein conjugate.

### DETAILED DESCRIPTION OF THE INVENTION

Definitions

As used herein, the term “adjuvant” refers to a substance added to a vaccine formulation to improve the immune response, for example, aluminum phosphate (see Singh et al., Proc. Natl. Acad. Sci. USA, Jan. 18, 2000 97(2): 811-6). Adjuvants include immunomodulators including, but not limited to, cytokines, such as IL-1B, TNFα, IL-2, IL-4, GM-CSF, IL-12, IL-1 8, to name a few, lymphokines, interleukins, interferons and growth factors. Cytokines, such as interferon-gamma, have found to have particular utility in tumor-cell vaccines. See van Slooten et al., Int. J. Pharm. (Jun. 10, 1999), 183(1): 33-6; see also van Slooten et al., Pharm. Res. (January 2000), 17(1): 42-8). Cytokines have also been found to enhance antiviral responses. See Barouch et al., Potent CD4⁺ T cell responses elicited by a bicistronic HIV-1 DNA vaccine expressing gp120 and GM-CSF. J Immunol. (2002) 168:562-568; see also Sun et al., Co-expression of granulocyte-macrophage colony-stimulating factor with antigen enhances humoral and tumor immunity after DNA vaccination. Vaccine (2002) 20:1466-1474.

Adjuvants also include proteins that are toxins. Studies have shown that genetically modified toxins can enhance immune responses.

Adjuvants also include chemokines, MPL and saponins (i.e., CSL). Suitable chemokines are disclosed in Ulrich H. von Andrian, M.D., Ph.D. and Charles R. Mackay, Ph.D. T-Cell Function and Migration: Two Sides of the Same Coin. Ian R. Mackay, M.D., and Fred S. Rosen, M.D., eds. The New England Journal of Medicine 343(14), 1020-1034, Oct. 5, 2000.

Adjuvants can be coadministered with the vaccine delivery system or may be incorporated into the vaccine complex. In DNA vaccines, for instance, the cDNA for a particular cytokine may be incorporated into the vaccine under control of a separate promote. Alternatively, the cytokine may be expressed as a fusion protein together with the M cell ligand and immunogen.

As used herein, the term “antibody” refers to an immunoglobulin molecule that has a specific amino acid sequence by virtue of which it interacts only with the antigen that induced its synthesis in cells of the lymphoid series (especially plasma cells) or with antigen closely related to it. Antibodies are classified according to their mode of action as agglutinins, bacteriolysins, haemolysins, opsonins, precipitins, etc. Antibodies would include not only antibodies raised during an in vivo immune response to antigen, but also those that are engineered or obtained in vitro, including human, humanized and chimeric antibodies, Fv and Fab₂ fragments, and immunologically reactive fragments thereof, such as the Fab and Fab′, of F(ab′)₂ fragments, etc.

As used herein, the term “antigen” refers to a substance recognized as foreign by the immune system and can be an immunogen.

As used herein, the term “complexed” refers to molecules that are non-covalently bound to each other through one or more linker molecules.

As used herein, the term “complexing agent” refers to a compound that is capable of non-covalently binding two molecules together.

As used herein, the term “conjugated” refers to molecules that are covalently bound to each other through one or more linker molecules.

As used herein, the term “crosslinker” refers to a compound that is capable of covalently binding two molecules together. After the reaction, the crosslinker, or part of the crosslinker, forms a part of the linkage between the conjugated molecules.

As used herein, the term “DNA vaccine” specifically refers to a therapeutic or prophylactic pharmaceutical formulation that contains a nucleic acid that encodes a protein or peptide against which a vaccinated host is induced to mount an immune response, preferably a protective immune response. Preferably, such a DNA vaccine contains a complete eukaryotic expression system encoding the molecular machinery for the expression of such a protein or peptide subunit vaccine. For example, such a DNA vaccine may be encoded in plasmid nucleic acids, which comprise promoters, enhancers, transcriptional terminators, etc. or any other sequences required for gene expression.

As used herein, the term “enteric adhesin” refers to a peptide, protein, carbohydrate or other class of compound that allows for or facilitates pathogen attachment to M cells as a means to gain entry to the host. For example, a reovirus al protein having a molecular weight of 47 kDa is an enteric adhesin (Nagata et al., Nucleic Acids Res (1984) 12(22):8699-710).

As used herein, the term “expression” refers to the expression of peptides or proteins that are encoded by, for example, the DNA vaccine or associated delivery vector. After expression of such a peptide or protein by, for example, an M cell to which a DNA vaccine has been targeted, such expression by the M cell would lead to the induction of an immune response by a vaccinated host against that encoded immunogen.

As used herein, the term “fusion protein” refers to a protein comprising a first polypeptide portion, for instance which functions to target such a protein to the mucosal lymphoid tissue, such as a polypeptide derived from an M cell ligand protein, which is operably linked to a second polypeptide portion, for instance which functions as a linker to couple the M cell ligand polypeptide to the immunogen to be targeted to the mucosal lymphoid tissue. Where the immunogen is a peptide, such a fusion protein may comprise an M cell ligand polypeptide operably linked to the immunogen peptide itself, without an intervening linker sequence. In this context, “operably linked” typically means that the fusion protein is expressed from a single mRNA that is expressed from a single gene sequence.

As used herein, the term “immunization” refers to a process that increases or enhances an organism's reaction to antigen and therefore improves its ability to resist or overcome infection. Immunization of animals may be used to obtain antibodies against pathogen epitopes to be used in epitope identification and library screening, for instance.

As used herein, the term “immunogen” refers to an antigen that is capable of eliciting (inducing) an immune response. For example, an immunogen usually has a fairly high molecular weight (usually greater than 10,000 daltons). Thus, for example, a variety of macromolecules such as peptides, proteins, lipoproteins, lipids, glycoproteins, polysaccharides, carbohydrates, some nucleic acids, and certain of the teichoic acids, can act as immunogens.

As used herein, the term “infectious agent” refers to a microorganism (or associated substance such as a toxin) that affects or communicates disease through invasion and multiplication of said substance in body tissues, which may be clinically unapparent or result in local cellular injury due to competitive metabolism, toxins, intracellular replication or antigen antibody response.

As used herein, the term “ligand” refers to any molecule that binds to another; in normal usage a soluble molecule such as a hormone or neurotransmitter, that binds to a receptor.

As used herein, the term “linker” refers to a moiety that brings two molecules into close enough association such that the two molecules may effectively be delivered together to a target cell. Linkers, for example, would include moieties that form complexes, conjugates, covalent and noncovalent associations as well as those that provide a carrier for an M cell targeting moiety and immunogen or an immunogen-encoding DNA.

As used herein, the term “M cell(s)” and “follicle associated epithelium” refer to specialized mucosal cells overlying mucosal associated lymphoreticular tissue (MALT), gut associated lymphoid tissue (GALT), bronchus associated lymphoid tissue (BALT) and nasal associated lymphoid tissue (NALT) and any other corresponding mucosal cells that are known to or become known to persons skilled in the art.

As used herein, the term “M cell specific ligand” refers to a molecule that selectively binds to a receptor available on the surface of follicle associated epithelial cell subpopulations, and an M cell specific physiologic effect accompanies that binding (e.g., uptake of pathogen). For example, the enteric adhesin, protein σ1 of reovirus, is an M cell specific ligand, as would be any M tropic portion or fragment of σ1 that retains the ability to selectively bind to follicle associated epithelial cell subpopulations. M cell tropic portions of protein a 1 are known in the art. For instance, Bassel-Duby et al. characterized the amino acid sequence of protein σ1 and defined a carboxy terminal portion of the protein as being responsible for receptor interaction (Nature, May-June 1985, 315(6018): 421-3). Similarly, by characterizing deletion mutants of protein σ1, Nagata et al. defined the receptor binding domain as being localized to two restriction fragment-generated domains in the carboxy terminus of the protein (Virology, September 1987, 160(1): 162-8). Nibert and colleagues found that there were two separate domains that contributed to receptor binding, one in the amino terminus and one in the carboxy terminus of protein ρ1 (J. Virol., August 1995, 69(8): 5057-67). Therefore, M cell-tropic variants of protein σ1 would also include variants with internal deletions but retaining both the amino and carboxy terminus. An M cell ligand of the invention would also include a tetramer or trimer of protein σ1 or variants of protein σ1, as σ1 has been reported to form tetramers and dimers in binding to cells (see Banerha et al., Virol. 167: 601-12 (1988); see also Strong et al., Virol. 184(l):23-32 (1991)).

By way of distinction, transferrin and certain other M cell lectins are not considered M cell specific ligands because: 1) the transferrin receptor is not limited to M cells (e.g., neurons express these receptors: Taylor et al., J Comp Physiol (1991) 161(5):521-524) and would not select for follicle associated epithelium subpopulations; and 2) because certain M cell lectins select for a:-linked galactose, and many cells possess carbohydrates with said linkages which are not follicle associated epithelium cells (e.g., hepatocytes: Oda et al., J Biol Chem (1988) 263(25):12576-12583). While M cell ligands (rather than M cell specific ligands) are contemplated for the compositions and methods of certain embodiments of the present invention, the M cell specific ligands are preferred.

As used herein, the term “minitopes” refers to peptide-size epitopes that are the “mimimum units” of structure of an antigenic biomolecule. The term “minigenes” refers to the correspondingly short oligonucleotide sequences that encode the “minitopes.”

As used herein, the term “mucosal” refers to any membrane surface in a host organism, preferably a mammal such as a human being or agriculturally important animal, that is covered by mucous.

As used herein, the term “nucleic acid” includes DNA and RNA molecules and is used synonymously with the terms “nucleic acid sequence” and “polynucleotide.” As used herein, the term “nucleic acid binding moiety” refers to compositions and substances that are capable of binding to or complexing with DNA and serving as a vehicle to attach the DNA to the M cell ligand-containing compositions of the present invention. Polybasic chains of amino acids are particularly contemplated for this purpose, as are, for example, synthetic compounds known to persons skilled in the art that have appropriate ionic charges to form complexes with DNA.

As used herein, “polymeric chain” refers to compounds formed by the joining of smaller, usually repeating, units linked by covalent bonds.

As used herein, the term “polymeric chain of basic amino acids” (i.e., polybasic) refers to a DNA binding sequence that is rich in basic amino acids, such as lysine, arginine, and ornithine, that is typically about ten to 300 residues long. D-isomers of these basic amino acids are suitable so long as the length of the stretch of basic amino acids is within the prescribed length. The polymeric chain of basic amino acids can be a homopolymer of a basic amino acid or it can comprise more than one kind of basic amino acid residue.

As used herein, “polypeptide” refers to an amino acid sequence including, but not limited to, proteins and protein fragments, naturally derived or synthetically produced.

As used herein, “protective immune response” refers to an immune system reaction that a human or animal develops in response to a vaccine that protects the human or animal against subsequent challenge with the pathogen or cancer from which the vaccine was designed. “Protects” in this sense means that the vaccinated human or animal develops a “memory” for the immunogen in the vaccine such that a prompt immune response occurs when the immunogen is later presented on or by a pathogen or tumor cell, and as a consequence the human or animal does not develop a disease caused by the pathogen or a malignancy associated with a vaccine tumor antigen.

As used herein, the term “reovirus” refers to a genus of the family Reoviridae infecting vertebrates only. Transmission is horizontal and infected species include humans, birds, cattle, monkeys, sheep, swine, and bats. Reovirus 1, reovirus 2, and reovirus 3 infect mammals, and reovirus 1 is the type species.

As used herein, the term “transcriptional factors” refer to a class of proteins that bind to a promoter or to a nearby sequence of DNA to facilitate or prevent transcription initiation.

As used herein, “tumor specific immunogens” refer to immunogens that are preferentially expressed by tumor cells, more preferably immunogens that are selectively expressed by tumor cells.

As used herein, the term “vaccination” refers to the introduction of vaccine into the body of an animal (or host) for the purpose of inducing immunity.

As used herein, the term “vaccine” generally refers to a therapeutic or prophylactic pharmaceutical formulation that contains a component against which a vaccinated host is induced to mount an immune response, preferably a protective immune response. For example, such a component could be a nucleic acid that is expressed by a vaccinated host to form an expressed protein or peptide subunit vaccine. Alternatively, such a component could be an immunogenic peptide or any other molecule that induces an immune response. “Therapeutic” vaccine means that the immune response raised by the vaccine treats or ameliorates or lessens an ongoing infection or cancer, for instance. “Prophylactic” means that the vaccine induces a protective immune response that protects the subject against a future infection or cancer.

General

Targeting of peptide and other epitopes and epitope minigenes to the mucosal immune compartments of the host is a main object of the invention. Mucosal inductive sites in humans, such as the Peyer's patches in the intestinal tract and the nasal-associated lymphoreticular tissue in the oropharyngeal cavity, stand as sentinels to the intestinal and respiratory tracts and represent the major sites where mucosal immune responses are initiated. Ghose et al., 1988 Mol. Immunol. 25(3):223-230.

The common cellular feature of these inductive sites are microfold or M cells scattered about a mucosal surface. (Neutra. et al; Cell (1996) 86: 345-348; Kermeis. et al., Gastroenterology (1993) 277: 949-952; van Ginkel et al., Merg. Infect. Dis. (2000) 6: 123-132). M cells appear to function in the uptake, transport, processing and presentation of microbial antigens. Thus, vaccine constructs that target epitopes and epitope DNA to respiratory or intestinal M cells represent the basic formulation for the development of new mucosal vaccines that can be administered by oral, rectal and nasal vaccines or by inhalation.

This invention provides DNA vaccines, preferably polybasic-M cell ligand conjugate-polynucleotide complexes, which, when directly introduced into a vertebrate in vivo, including mammals such as humans, induce the expression of encoded proteins within the animal. Prior to the present invention, the art had taught that DNA vaccines represent an efficient method of inducing immunity against a given pathogen if the responsible gene for eliciting protection is identified. As described below, the present inventors have found that the described DNA vaccine formulations improve the targeting of DNA to mucosal inductive tissues. The present invention is based, in part, on the ability of such vaccine formulations to selectively and preferentially target mucosal inductive tissues. Mucosal inductive tissues are sites within the mucosa that support the development of B and T lymphocytes to become stimulated against a specific pathogen or vaccine component or subunit. If the antigen or vaccine can reach this site, there is a strong likelihood that a mucosal immune response will be induced.

The invention also provides other immunogen complex vaccines, preferably M cell ligand conjugate-immunogen complexes, where the immunogen is conjugated to the M cell ligand by any suitable moiety. Such vaccine formulations are also designed to selectively and preferentially target mucosal inductive tissues by virtue of the M cell ligand portion of the complex. Once the complexed immunogen is delivered to the target mucosal tissue, a mucosal immune response will be induced.

To specifically induce such a mucosal immune response, the compositions and methods of the present invention employ ligands formulated to preferentially or specifically target the specialized epithelium that surrounds mucosal inductive tissues referred to as M cells. Thus, with regard to DNA vaccines, an M cell ligand binds M cells to mediate internalization of the attached DNA. With regard to other types of vaccines including those comprising complexed peptide and carbohydrate immunogens, an M cell ligand binds M cells to mediate localization of the immunogen to the mucosal lymphoid tissue. In one embodiment, the M cell ligand is an adhesin of a pathogen, preferably an enteric adhesin of a pathogen, such as σ1 protein of a reovirus. Additionally, adhesins from Salmonella and poliovirus, as well as other infectious agents having the same tissue tropism would be appropriate. See Frey et al., Behring Inst. Mitt. (February 1997) 98: 376-89; Sansonetti & Phalipon, M cells as ports of entry for enteroinvasive pathogens: mechanisms of interaction, consequences for the disease process, Semin. Immunol. (1999) 11:193-203; Wilson et al., Salmonella enterica serovars gallinarum and pullorum expressing Salmonella enterica serovar typhimurium type 1 fimbriae exhibit increased invasiveness for mammalian cells, Infect Immun. (2002) 68:4782-4785; Neutra, Interactions of viruses and microparticles with apical plasma membranes of M cells: implications for human immunodeficiency virus transmission, J Infect Dis. (1999) 179 Suppl 3:S441-S443. For example, the nucleotide sequences encoding said proteins include but are not limited to polynucleotides comprising nucleotide sequences as set forth in GenBank accession numbers: J02325; M10491; AF059719; AF059718; AF059717; AF059716; U74293; and U74292.

In another embodiment, the M cell ligand may be an enteric adhesin of a pathogen such as an intimin of an enteropathic Escherichia coli. For example, the nucleotide sequences encoding said intimin protein include but are not limited to polynucleotides comprising nucleotide sequences as set forth in GenBank accession numbers: U38618; AJ223063; Y13111; Y13112; AF043226; and U62657. In another embodiment, the immunogen is an enteric adhesin receptor of a pathogen such as an Tir of an enteropathic Escherichia coli. For example, the nucleotide sequences encoding the intimin receptor protein include but are not limited to polynucleotides comprising nucleotide sequences as set forth in accession number: AF113597. In another embodiment, the immunogen is an enteric adhesin of a pathogen such as an invasin of Salmonella typhimurium, Yersinia pestis and pseudotuberculosis and enteropathic Escherichia coli. For example, the nucleotide sequences encoding said invasin proteins include but are not limited to polynucleotides comprising nucleotide sequences as set forth in accession numbers: AF140550; Z48169; X53368; U25631; and M17448.

In another embodiment, the immunogen may be a peptide mimetic, or a minitope, of an infectious agent or a tumor specific antigen, or the nucleotide sequence encoding the minitope being termed a minigene. The invention relates to novel technology for developing vaccine constructs capable of eliciting long-term systemic and mucosal immune responses to diverse epitope structures including conformational epitopes embedded in complex structures of infectious agents. This method emphasizes, in a preferred embodiment, the selection of peptide mimics and code (nucleotide sequence) for antibody binding domains displayed in a library of modularly coded biomolecules in a phage display format. (Burritt. et al.Anal. Biochem. 238: 1-13). Display technology has become a routine tool for enriching molecular diversity and producing novel types of proteins or peptides (Li. Nat. Biotechnol. (2000) 12: 1251-1256). The use of display technology circumvents the need for genomic analysis and the difficulty, indeed, the impossibility in some cases, of deducing from gene sequence analyses the structure and antigenic properties of epitopes containing protein, lipid and carbohydrate or formed as a discontinuous sequence of amino acids or monosaccharides. (Scott. Et al., Science (1990) 249: 386-390).

Particularly contemplated are nucleotide and other vaccines in which the immunogen to be delivered to the target mucosal tissue is an immunogen expressed by an infectious agent such as a microorganism or is a tumor specific antigen. Preferred immunogens are derived from or, like an expressed toxin, are associated with a bacterium, protozoan, parasite, virus, fungus, prion, tuberculobacillus, leprosy bacillus, malaria parasite, diphtheria bacillus, tetanus bacillus, Leishmania, Salmonella, Schistosoma, measles virus, mumps virus, herpes virus, HIV, cancer and influenza virus.

Exemplary bacterial disease organisms include: Group A streptococci, Group B streptococci, Streptococcus faecalis, Staphylococcus aureus, Listeria monocytogenes, Helicobacter pylori, Bacillus anthracis, Brucella abortus, Brucella melitensis, Neisseria gonorrhoeae, Neisseria meningitidis, Hemoplilus influenzae, Mycobacterium tuberculosis, Bordetella pertussis, Vibrio cholerae, Salmonella typhi, Salmonella enteritidis, Shigella dysenteriae, Shigella flexneri, Escherichia coli 0157:H7, Escherichia coli, Escherichia coli (bovine scouring strains), Chlamydia pneumoniae and Chlamydia trachomatis.

Exemplary bacterial toxins and microorganisms include: A/B bacterial toxins, such as Shiga toxin-Shigella, Shiga-like toxins-Enterohemorrhagic E. Coli, Diptheria toxin-Corynebacterium diptheriae, Botulinum toxin-Clostridium botulinum, Tetanus toxin-Clostridium tetani, Cholera toxin-Vibrio cholerae, A toxin-Pseudomonas aeruginosa, LT-ETEC-Escherica coli; α, β, χ, δ, ε,η, θ, κ, γ toxin-Clostridium perfringes; Dick (Erythrogenic) toxin-Streptococcus pyrogenes; Lethal toxin-Bacillus anthracis; Alpha toxin-Staphylococcus aureus; and Plague toxin-Yersinia pestis. Fungal diseases include: Candida albicas; Aspergillus fumigatus; Cryptococcus neoformans; Coccidioides immitis; and Histoplasma capsulatum.

Exemplary viral diseases and causative agents include: Rhinoviruses-polio, cold viruses; Alphaviruses-yellow fever, encephalitis; Lyssavirus-rabies; Calcivirus-norwalk virus; Prthopox virus-smallpox; Papillomavirus-warts; HIV; HPV; Herpesvirus-genital herpes, simplex, shingles, chickenpox; Bunyavirus-hentavirus; Coronavirus-respiratory infections; Mobillivirus-mumps, measles; Reovirus-respiratory infections; Enterovirus-intestinal infections; Influenzavirus-influenza.

Exemplary spirochetal diseases and organisms include Treponema pallidum (syphilis) and Borrelia recurrentis (Recurring fever). Exemplary protozoan diseases and causative agents include: Entamoeba histolytica; Giardia lamblia; Taxoplamsa gondii; Plasmodium species (Plasmodium); Trypanosoma cruzi; Trypanosoma gambiiense; Leishmaniasis donovani; Pneumocystis carinii, Cryptosporidium, Trichomonas vaginalis; Schistosoma mansoni and Tritrichomonas faetus.

Exemplary antigens to be included in whole or in part as suitable immunogens, or to be encoded by the nucleotide vaccines of the invention, and the diseases with which they are associated include, but are not limited to: tuberculosis (e.g., BCG antigen: Kumar et al., Immunology (1999) 97(3):515-521), leprosy (e.g., antigen 85 complex: Naito et al., Vaccine (1999) 18(9-10):795-798), malaria (e.g., surface antigen MSA-2: Pye et al., Vaccine (1997) 15(9):1017-1023), diphtheria (e.g., diphtheria toxoid: U.S. Pat. No. 4,691,006 ), tetanus (e.g., tetanus toxin: Fairweather et al., Infect Immun (1987) 55(11):2541-2545), leishmania (e.g., Leishmania major promastigotes: Lasri et al., Vet Res (1999) 30(5):441-449), salmonella (e.g., covalently bound capsular polysaccharide (Vi) with porin, both isolated from S. typhi.: Singh et al., Microbiol Immunol (1999) 43(6):535-542), schistomiasis (e.g., major antigen of Schistosoma mansoni (Sm28 GST): Auriault et al., Pept Res (1991) 4(1):6-1 1), measles (e.g., the surface glycoprotein and fusion protein of measles virus: Machamer et al., Infect Immun (1980) 27(3):817-825), mumps (e.g., hemagglutinin-neuraminidase (HN) viral gene product: Brown et al., J Infect Dis (1996) 174(3):619-622), herpes (e.g., HSV-2 surface glycoproteins (gB2 and gD2): Corey et al., JAMA (1999) 282(4):331-340), AIDS (e.g., gp160: Pontesilli et al., Lancet (1999) 354(9182):948-949), influenza (e.g., immunodominant peptide from hemagglutinin: Novak et al., J Clin Invest (1999) 104(12):R63-67), Group A streptococcus: (extracellular cysteine protease, Lukomski, S. et al., Infect. Immun. 1999. 67(4): 179-1788, Streptococcal inhibitor of complement (Sic) (Lukomski, S., et al., Infect. Immun. 2000. 68(2): 535-542, Hyaluronic acid capsule, Schrager, H. et al., J. Clin. Invest. 1998. 101: 1708-1716), Group B streptococcus (capsular polysaccharide, Type I, II, II, IV and V, Pincus, S. H, et al., J. Immunol. 1998. 160: 293-298.), Shigella species (Lipopolysaccharide (0 somatic antigen) Phalipon, A., et al., Eur. J. Immunol. 1997. 27: (10), 2620-2625), Brucella abortus (Lipopolysaccharide (antigen A) Montaraz, J., et al., Immun. 1986. 51: 961-963), Escherichia coli (EPEC) (intimin,and/or Hp90protein, Hartland, et al., Mol. Microbiol. 1999. 32 (1): 151-158, Kenny, B., et al., Cell. 1997. 91: 511-520), Escherichia coli (EHEC) 0157-H7 (lipopolysaccharide (LPS), Konadu, E., et al., Infect. Immun. 67:6191-6193), Salmonella typhi (Vi capsular polysaccharide, Singh, et al., Microbiol. Immunol. 1999. 43(6): 535-542), Vibrio cholerae (cholera toxin B subunit, Liljeqvist, S., et al., Appl. Environ. Micro. 1997. 63(7): 2481-2488), Helicobacter pylori (Urease A and B, Lee, C. et al. J. Infect. Dis. 1995. 172: 161-172, Le ^{b} binding adhesin, Iver, D. et al., Science, 1998. 279: 373-377), Bordetella pertussis (Filamentous hemagglutinin (FHA), Brennan, M. and R. Shahin. Am. J. Respir. Crit. Care Med. 1996, 154:145-149), Haemophilus influenze (HMW1/HMW2 adhesin, St. Geme, J. The Finnish Med. Soc. DUODECIM. Ann. Med. 1996, HifE pilus (adhesin), Hia adhesin, Barenkamp, S and J. St. Geme, Mol. Microbiol. 1996. 19: 1215-1223), Chlamydia peumoniae (Major outer membrane protein (MOMP), Peterson, E., et al., Infect. Immun., 1996. 64(8): 3354-3359), HIV (Fusion-dependent immunogen, LaCasse, R. A., et al., Science. 1999. 283: 357-362, 5-Helix protein, Root, M. et al., Sciencexpress Report, Jan. 11, 2001), Poliovirus (M cell ligand, Frey, A. et al., Behring Inst. Mitt. 1997. 98: 376-389), Measles virus (surface glycoprotein, fusion protein, Machamer et al., Infect. Immun. 1980. 27(3): 817-825), Cryptococcus neoformans (Capsular polysaccharide-glucuronoxylomannan, Blackstock, R. and A. Casadevall. 1997. Immunol. 92:334-339), and Schistosoma mansoni (9B antigen peptides,Arnon, R. et al., Immunology. 101(4): 555-562). Administration of such antigens in formulations according to the invention to a host results in stimulation of the host's immune system to produce a protective immune response.

Exemplary tumor specific antigens may be derived from cancers including: leukemia-lymphocytic, granulocytic, monocytic or myelocytic; Lymphomas; basal cell carcinoma; squamous cell carcinoma; breast, colon, endometrial, pancrecatic, lung, etc. carcinoma; and uterine, vaginal, prostatic, testis, ostogenic or pulmonary sarcoma (see Wang R F., J Mol Med (1999) 77(9):640-655). Tumor antigens according to the invention include 707-AP (707 alanine proline), AFP (alpha (α)-fetoprotein), ART-4 (adenocarcinoma antigen recognized by T cells 4), BAGE (B antigen), β-catenin/m (β-catenin/mutated), Bcr-abl (breakpoint cluster region-Abelson), CAMEL (CTL-recognized antigen on melanoma), CAP-1 (carcinoembryonic antigen peptide-1), CASP-8 (caspase-8), CDC27m (cell division-cycle 27 mutated), CDK4/m (cycline-dependent kinase 4 mutated) CEA (carcinoembryonic antigen), CT (cancer/testis antigen), Cyp-B (cyclophilin B), DAM ((differentiation antigen melanoma) (the epitopes of DAM-6 and DAM-10 are equivalent, but the gene sequences are different; DAM-6 is also called MAGE-B2 and DAM-10 is also called MAGE-B1), ELF2M (elongation factor 2 mutated), ETV6-AML1 (Ets variant gene 6/acute myeloid leukemia 1 gene ETS), G250 (glycoprotein 250), GAGE (G antigen), GnT-V (N-acetylglucosaminyltransferase V), Gp100 (glycoprotein 100 kD), HAGE (helicose antigen), HER 2/neu (human epidermal receptor-2/neurological), HLA-A*0201-R170I (arginine (R) to isoleucine (I) exchange at residue 170 of the α-helix of the α2-domain in the HLA-A2 gene), HPV-E7 (human papilloma virus E7), HSP70-2M (heat shock protein 70-2 mutated), HST-2 (human signet ring tumor-2), hTERT or hTRT (human telomerase reverse transcriptase), iCE (intestinal carboxyl esterase KIAA0205 (name of the gene as it appears in databases), LAGE (L antigen), LDLR/FUT (low density lipid receptor/GDP-L-fucose: β-D-galactosidase 2-α-L-fucosyltransferase), MAGE (melanoma antigen), MART-1/Melan-A (melanoma antigen recognized by T cells-1/Melanoma antigen A), MC1R (melanocortin 1 receptor), Myosin/m (myosin mutated), MUC1 (mucin 1), MUM-1 , -2, -3 (melanoma ubiquitous mutated 1, 2, 3), NA88-A (NA cDNA clone of patient M88), NY-ESO-1=New York-esophageous 1), P15 (protein 15),p190 minor bcr-abl (protein of 190 KD bcr-abl), Pml/RARα (promyelocytic leukaemia/retinoic acid receptor α), PRAME (preferentially expressed antigen of melanoma), PSA (prostate-specific antigen), PSM (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), WT1 (Wilms' tumor gene). These antigens are disclosed in references that are cited in Cancer Immunology Immunotherapy 50:3-15 (2001), which is herein incorporated by reference. The cited references may be consulted for methods of isolating the specific antigens or genes encoding the specific antigens for use in the vaccines of the invention.

In general, it is the formulation of an appropriate DNA conjugate or immunogen complex (or other delivery vector) to deliver the DNA to a target M cell that improves host immune responses against a specific pathogen or other immunogen. For example, such a vaccine may be comprised of a polybasic conjugate/DNA complex by incorporating an M cell ligand. Thus, for any given immunogen encoded by a nucleic acid or mimicked by a peptide encoded by a nucleic acid that can be used for eliciting a host response, such a response can be enhanced through effective targeting mediated by M cell ligands.

DNA Conjugate Vaccines

M cell-directed-DNA vaccine formulations of the present invention have been demonstrated to be a robust epitope DNA delivery technology. In one embodiment, the formulations employ plasmid DNA (pDNA) constructs (Shroff et al., Pharm. Sci. Tech. Today (1999) 2: 205-212), that can be designed to contain either a single epitope or multiple epitopes (polytopes). Thus, many different formulations of epitope DNA-M cell directed vaccines which target epitope DNA and immunogenic peptides to key compartments of the immune system can be used to elicit potent cellular as well as humoral immune responses to infectious agents. There is every expectation that the same technology could be applied to the development of vaccines against many forms of cancer (Kieber-Emmons et al., J. Immunol. (2000) 165:623-627; Qui et al., Hybridoma (1999) 18:103-112), parasitic diseases, (Arnon et al., Immunol. (2000) 101:555-562; de la Cruz. et al.J. Biol. Chem. (1988) 283: 4318-4322) viral diseases, (Frey. et al., Behring Inst. Mitt. (1997) 98: 376-389: Prince. et al., Vaccine. (1997) 15: 916-919) fungal diseases, (Glee. et al., Ann. Meeting Am. Soc. Micro. (1997), and bacterial infections (Lowrie. et al., Springer Semin. Immunolpathol., (1997) 19: 161-173; Wu et al., Gene Therapy (2000) 7:61-69).

The observation that mammalian cells can express genes encoded by plasmid DNA (pDNA) injected intramuscularly has led to experiments using gene immunization. (Donnelly. et al Ann. Rev. Immunol (1997) 15: 617-648; Shroff, et al., Pharm./Sci. Tech Today. (1999) 2: 205-212; Lee. et al., Ann. Med. (1998) 30: 460-468; Jones. et al., Vaccine. (1997) 115: 814-817). The pDNA of vaccine constructs has not been found to integrate into host chromosomal DNA (unlike retrovirus and adenovirus vectors) thereby averting the activation of oncogenes or disruption of normal gene function, nor do pDNA preparations (if free of protein and RNA) stimulate an anti-DNA humoral response or generate tolerance in neonatal animals.

DNA vaccines can be delivered by any route but the most promising appears to be those that target key cells (antigen-presenting/processing cells) in immune compartments of the mucosal membrane. To be expressed in a host cell, plasmids must cross the plasma membrane, escape endosomal degradation pathways and access the cytoplasm. By any method of administration, pDNA must finally enter the nucleus before gene expression can commence, but, once there, normal cellular transcriptional and translational pathways are exploited for the production of gene products. The enormous potential of DNA vaccines lies in their versatility, ease of manufacture and safety. Of paramount importance is their ability to induce a cytotoxic T lymphocyte (CTL) response, which is necessary for the eradication of most viral diseases, certain bacterial diseases (tuberculosis, Brucella), parasitic infections and tumors. (Jones. et al., Vaccine. (1997) 15: 814-817; Offit. et al., J. Virol. (1991) 65: 1318-1324; Gallichan. et al., J. Exp. Med. (1996) 184: 1879-1890; Hilleman, M. et al., Vaccine (1998) 16: 778-793; Van Ginkel et al., Emerg. Infect. Dis. (2000) 6(2)123-132). The ability to stimulate a CTL response coupled with the capacity for repeated (endogenous) immunizations also raises the possibility of using these vaccines for therapy.

Our presently formulated DNA vaccines induce improved mucosal IgA antibody responses and promote sustained CTL responses, demonstrating efficacious vaccination via the mucosa. Further, as the present invention shows the ability of the protein σ1 to mediate efficient gene transfer to the nasal-associated lymphoid tissue (NALT) in vivo, we have demonstrated that systemic and mucosal immunity to intranasally delivered DNA or peptides or other antigens as part of an M cell ligand complex is achievable.

In a preferred embodiment, a contemplated polynucleotide is a nucleic acid which contains essential regulatory elements such that upon introduction into a living vertebrate cell, it is able to direct the cellular machinery to produce translation products encoded by the structural gene sequence component of the polynucleotide. In one embodiment of the invention, the polynucleotide is a polydeoxyribonucleic acid comprising immunogen (or antigen) structural genes or fragments thereof operatively linked to a transcriptional promoter(s). In another embodiment of the invention the polynucleotide comprises polyribonucleic acid encoding antigen structural genes or fragments thereof which are amenable to translation by the eukaryotic cellular machinery (ribosomes, tRNAs, and other translation factors).

Where the protein encoded by the polynucleotide is one which does not normally occur in that animal except in pathological conditions, (i.e. an heterologous protein) such as proteins associated with human immunodeficiency virus (HIV) and Brucella, the animals' immune system is activated to launch a protective immune response. Because these exogenous proteins are produced by the animals' own tissues, the expressed proteins are processed by the major histocompatibility system (MHC) in a fashion analogous to when an actual infection occurs. The result, as shown in this disclosure, is induction of immune responses against an antigen.

Polynucleotides for the purpose of generating immune responses to an encoded protein are referred to herein as polynucleotide or DNA vaccines. The described vaccine works by inducing the vaccinated animal to produce antibodies or cell-mediated immune responses specific for the vaccine. The production of these antibodies or cell-mediated immune responses will protect the host upon subsequent exposure to the infectious agent.

The present invention further provides recombinant DNA molecules (rDNAs) that contain a coding sequence. The vaccines are produced using conventional eukaryotic plasmid expression systems for the encoded gene. As used herein, a rDNA molecule is a DNA molecule that has been subjected to molecular manipulation in situ. Methods for generating rDNA molecules are well known in the art, for example, see Sambrook et al., Molecular Cloning (1989). In the preferred rDNA molecules, a coding DNA sequence is operably linked to expression control sequences and/or vector sequences.

The choice of vector and/or expression control sequences to which one of the protein encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a host cell's environment.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from Biorad Laboratories, (Richmond, Calif.), pPL and pKK223 available from Pharmacia, Piscataway, N.J.

Expression vectors compatible with eukaryotic cells, preferably those compatible with vertebrate cells, can also be used to form a rDNA molecules that contains a coding sequence. Eukaryotic cell expression vectors are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the DNA vaccine molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al, J. Mol. Anal. Genet. 1:327-341, 1982.) Alternatively, the selectable marker can be present on a separate plasmid, and the two vectors are introduced by co-transfection of the host cell, and selected by culturing in the appropriate drug for the selectable marker.

The M cell ligand-polybasic conjugates employed in the DNA vaccines of the invention may be produced chemically or by the recombinant method. Coupling by the chemical method can be carried out in a manner known per se for the coupling of peptides and if necessary the individual components may be provided with linker substances before the coupling reaction (this procedure is necessary when there is no functional group suitable for coupling available at the outset, such as a mercapto or alcohol group).

Depending on the desired properties of the conjugates, particularly the desired stability thereof, coupling may be carried out by means of various techniques known to persons skilled in the art, including but not limited to the following techniques. For example, the use of disulphide bridges, which can be cleaved again under reductive conditions (e.g., using succinimidyl pyridyl dithiopropionate, are contemplated. See Jung et al., Biochem Biophys Res Comm 101:599-606 (Jul. 30, 1981). Also contemplated is the use of compounds which are largely stable under biological conditions (e.g., thioethers, by reacting maleimido linkers with sulfhydryl groups of the linker bound to the second component). Further comtemplated is the use of bridges that are unstable under biological conditions, e.g., ester bonds, or using acetal or ketal bonds which are unstable under weakly acidic conditions.

The production of the conjugates according to the invention by the recombinant method offers the advantage of producing precisely defined, uniform compounds, whereas chemical coupling produces conjugate mixtures which then have to be separated.

The recombinant preparation of the conjugates according to the invention can be carried out using methods known for the production of chimeric polypeptides. The present invention further provides methods for producing a protein of the invention using nucleic acid molecules herein described. In general terms, the production of a recombinant form of a protein typically involves the following steps:

First, a nucleic acid molecule is obtained that encodes an M cell ligand protein of the invention. If the encoding sequence is uninterrupted by introns, it is directly suitable for expression in any host. The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

The polybasic and other binding or linker moiety components may vary in terms of their size and amino acid sequence. Production by genetic engineering has the advantage of allowing the M cell ligand component of the conjugate to be modified, by increasing the ability to bind to the receptor, by suitable mutations, for example, or by shortening the M cell ligand component to the part of the molecule which is responsible for the binding to the receptor. It is particularly expedient for the recombinant preparation of the conjugates according to the invention to use a vector which contains the sequence coding for the M cell ligand component as well as a polylinker into which the required sequence coding for the polybasic or binding moiety component is inserted. In this way, a set of express plasmids can be obtained, of which the plasmid containing the desired sequence can be used as necessary in order to express the conjugate according to the invention.

The nucleic acids which are to be transported into the cell may be DNAs or RNAs, with no restrictions as to the nucleotide sequence. The nucleic acids may be modified, provided that this modification does not affect the polyanionic nature of the nucleic acids; these modifications include, for example, the substitution of the phosphodiester group by phosphorothioates or the use of nucleoside analogues.

With regard to the size of the nucleic acids the invention again permits a wide range of uses. There is no lower limit brought about by the transporting system according to the invention; thus, any lower limit which might arise would be for reasons specific to the particular intended use or target specificity. It is also possible to convey different nucleic acids into the cell at the same time using the conjugates according to the invention.

Within the scope of the present invention it has been possible to demonstrate that M cell ligand-polybasic conjugates can be efficiently absorbed into living cells and internalized. The disclosed conjugates or complexes according to the invention are not apparently harmful to cell growth. This means that they can be administered repeatedly and thus ensure a constantly high expression level of the genes and nuclectide sequences inserted into the cell.

The ratio of nucleic acid to conjugate can vary within a wide range, and it is not absolutely necessary to neutralize all the charges of the nucleic acid. This ratio will have to be adjusted for each individual case depending on criteria such as the size and structure of the nucleic acid which is to be transported, the size of the polybasic component and the number and distribution of its charges, so as to achieve a ratio of transportability and biological activity of the nucleic acid which is favorable to the particular application. This ratio can first of all be adjusted coarsely, for example by using the delay in the speed of migration of the DNA in a gel (e.g., using the mobility shift on an agarose gel) or by density gradient centrifugation. Once this provisional ratio has been obtained, it may be expedient to carry out transporting tests with the radioactively labeled complex with respect to the maximum available activity of the nucleic acid in the cell and then reduce the proportion of conjugate if necessary se that the remaining negative charges of the nucleic acid are not an obstacle to transportation into the cell.

The preparation of the M cell ligand-polybasic conjugate/nucleic acid complexes, which are also a subject of the invention, can be carried out using methods known per se for the complexing of polyionic compounds. One possible way of avoiding uncontrolled aggregation or precipitation is to mix the two components together first of all at a high (about 1 molar) concentration of common salt and subsequently to adjust to physiological saline concentration by dialysis or dilution. Preferably, the concentrations of DNA and conjugate used in the complex forming reaction are not too high (more than 100 μg/ml). to ensure that the complexes are not precipitated, as would be known to persons skilled in the art.

A preferred nucleic acid component of the M cell ligand-polybasic moiety-nucleic acid complex according to the invention is an immunogen structural gene. The invention further relates to a process for introducing nucleic acid or acids into human or animal cells, preferably forming a complex which is soluble under physiological conditions.

There are many embodiments of the instant invention which persons skilled in the art can appreciate from the specification. Thus, different transcriptional promoters, terminators, carrier vectors or specific gene sequences may be used successfully. Various methods are known for such constructs which, upon introduction into mammalian cells, induces the expression, in vivo, of the polynucleotide thereby producing the encoded protein. It is readily apparent to those skilled in the art that variations or derivatives of the nucleotide sequence encoding a protein can be produced which alter the amino acid sequence of the encoded protein.

It is well known in the biological arts that certain amino acid substitutions can be made in protein sequences without affecting the function of the protein. Generally, conservative amino acids are tolerated without affecting protein function. Similar amino acids can be those that are similar in size and/or charge properties, for example, aspartate and glutamate, and isoleucine and valine are both pairs of similar amino acids. Similarity between amino acid pairs has been assessed in the art in a number of ways. For example, Dayhoff et al. (1978) in Atlas of Protein Sequence and Structure, Volume 5, Supplement 3, Chapter 22, pages 345-352, which is incorporated by reference herein, provides frequency tables for amino acid substitutions which can be employed as a measure of amino acid similarity. Dayhoff et al. 's frequency tables are based on comparisons of amino acid sequences for proteins having the same function from a variety of different evolutionary sources. The altered expressed protein may have an altered amino acid sequence, yet still elicits immune responses which react with the antigen protein, and are considered functional equivalents. In addition, fragments of the full length genes which encode portions of the full length immunogenic protein may also be constructed. These fragments should encode a protein or peptide which elicits antibodies that cross-react with the immunogenic protein, and are considered to be functional equivalents.

Other M Cell-Immunogen Vaccines

Another embodiment of the present invention includes an M cell specific ligand or M cell tropic fragment or portion thereof conjugated or complexed to an immunogen, optionally via an appropriate linker. Immunogens in this instance would include a variety of macromolecules such as peptides, proteins, lipoproteins, lipids, glycoproteins, polysaccharides, carbohydrates, some nucleic acids, genes, and certain of the teichoic acids, or any other molecule from a pathogen or tumor cell that could be used to generate a protective immune response. Particularly preferred immunogens are as provided above.

Immunogens may be conjugated or complexed with the M cell specific ligand using any means known in the art. For instance, peptide and protein immunogens may be comprised in fusion proteins where they are operably linked to the M cell specific ligand or fragment. Fusion proteins are expressed from a single open reading frame encoding both the M cell specific ligand and the immunogen in such a manner that the M cell specific ligand portion retains its capability to target M cells and the immunogen retains its immunogenic potential. Fusion proteins can optionally contain an intermediate peptide region or linker connecting the M cell binding portion to the immunogen portion. Genes encoding the fusion proteins of the invention are also encompassed, as are plasmid vectors and host cells comprising and expressing the same.

Alternatively, immunogens may be conjugated or complexed with an M cell specific ligand using an appropriate linker. Such linkers may include chemical cross-linkers or fusion mediators. Cross-linking may be performed with either homo- or heterobifunctional agents, i.e., SPDP, DSS, SIAB. Methods for cross-linking are disclosed in PCT/DK00/00531 (WO 01/22995) which is herein incorporated by reference. Such methods may generally include the steps of:

a) reacting an antigen or immunogen with a first crosslinker thereby producing a mixture of crosslinker derivatives of the immunogen;

b) isolating the antigen derivatised with a single crosslinker residue,

c) activating the isolated crosslinker derivative of the antigen,

d) reacting an M cell ligand with a second crosslinker thereby producing a mixture of crosslinker derivatives of the M cell ligand component,

e) reacting the activated crosslinker derivative of the antigen with the mixture of crosslinker derivatives of the M cell ligand, thereby producing conjugates between the antigen and the M cell ligand.

In one embodiment, the first crosslinker is a bifunctional crosslinker (i.e., with two functional groups), preferably a heterobifunctional crosslinker (i.e., with two different functional groups). In another embodiment, the second crosslinker is a bifunctional crosslinker, preferably a heterobifunctional crosslinker. In a further embodiment, the first and/or second crosslinker is selected from the non-limiting group consisting of N-succinimidyl (4-lodoacetyl) aminobenzoate (SIAB), N-succinimidyl-3-(2-pyridylthio)propionate (SPDP), N-succinimidyl S-acetylthioacetate (SATA), m-maleimidobenzoyl-N-hydroxysuccinimide ester (MBS) and N-g-maleimidobutyryloxy-succinimide ester (GMBS). In a further embodiment, the first and/or second crosslinker is Traut's Reagent 2-iminothiolane in combination with SPDP. In still a further embodiment the first and/or second crosslinker is succinimidyl dicarbonyl pentane or disuccinimidyl suberate. In a further embodiment, the first and/or second crosslinker is selected among those disclosed in The Pierce Products Catalogue (Pierce Chemical Company, USA) and the Double Agents™ Cross-Linking Reagents Selection Guide (Pierce Chemical Company), which are herein incorporated by reference.

In the general method presented above, any suitable method may be used to purify the crosslinker derivatised immunogen. For instance, the crosslinked immunogen may be purified by preparative reverse phase HPLC (RP-HPLC). In another embodiment, the crosslinked immunogen may be purified by membrane filtration, such as ultrafiltration or diafiltration. Unreacted crosslinker may be removed by size exclusion chromatography, such as gel filtration. The final conjugate may also be purified using any suitable means, including for instance gel filtration, membrane filtration, such as ultrafiltration, or ion exchange chromotography, or a combination thereof.

Molar ratios to be used in crosslinking methodology may be readily optimized by those of skill in the art, but generally will vary between about 1:1 to about 5:1 crosslinker to immunogen or ligand depending on the crosslinker and the efficiency of crosslinking. The ratio of crosslinked immunogen to crosslinked M cell ligand to be admixed may also be readily optimized by those of skill in the art, but will generally range from about 1:1 to about 10:1 immunogen to M cell ligand.

Conjugates and complexes can comprise the following scenarios: polypeptides with attached immunogens may be conjugated to M cell specific ligands; liposomes can replace the polypeptide, wherein the M cell specific ligand may be conjugated to a liposome containing the immunogens, or conjugated to a liposome with one or several copies of an immunogen or different immunogens attached/displayed to its surface; and peptide and protein immunogens may be expressed as fusion proteins operably linked to the M cell specific ligand. Fusion proteins are known in the art, such as those disclosed in Yu et al., The biologic effects of growth factor-toxin conjugates in models of vascular injury depend on dose, mode of delivery, and animal species, J Pharm Sci. November 1998; 87(11): 1300-4; McDonald et al., Large-scale purification and characterization of recombinant fibroblast growth factor-saporin mitotoxin, Protein Expr Purif. August 1996; 8(1):97-108; Lappi et al., Expression and activities of a recombinant basic fibroblast growth factor-saporin fusion protein, J Biol Chem. Apr. 29, 1994;269(17):12552-8; and Prieto et al., Expression and characterization of a basic fibroblast growth factor-saporin fusion protein in Escherichia coli. Ann NY Acad Sci. (1991) 638:434-7. By way of example, fusion-derived immunogen conjugates include K99 fimbrial protein from bovine enterotoxigenic E. coli fused to protein σ1, colonization factor antigen 1 fimbrial protein from human enterotoxigenic E. coli fused to protein σ1 or myelin basic protein fused to protein σ1.

Liposomes are one means by which M cell ligands may be attached to immunogens. Liposomes may be made by means that are well known in the art, and may be polymerized or unpolymerized, depending on the desired characteristics for the liposome. In general, polymerized lipid compositions may be produced according to techniques described in U.S. Pat. No. 5,962,422 to Nagy et al., “Inhibition of Selectin Binding”, utilizing the materials and methods disclosed therein. Other suitable methods are disclosed in U.S. Pat. No. 6,342,226 to Betbeder et al., “Method for Increasing Immunogenicity, Product Obtained and Pharmaceutical Compositions”; U.S. Pat. No. 6,090,406 to Popescu et al., “Potentiation of Immune Responses with Liposomal Adjuvants”; and U.S. Pat. No. 6,225,445 to Shen et al., “Methods and Compositions for Lipidization of Hydrophilic Molecules.” In liposomal formulations, protein σ1 or another M cell targeting ligand is attached covalently or by other means to the liposome. The immunogen being delivered to the M cells may be either encapsulated within the liposome, such as for delivery of an immunogen which needs to be protected from interaction in vivo prior to the destination of choice, or displayed on the surface of the liposome. Adjuvants such as the immunomodulators described herein may be readily included in the liposome formulation.

Non-DNA vaccine formulations may work by receptor-mediated endocytosis pathways, wherein immunogens are associated with MHC molecules and displayed on the cell surface of follicle-associated epithelial cells. Non-DNA immnunogens may also mount a mucosal immune response by virtue of being targeted to the mucosal lymphoid tissue by means of an M cell ligand. The success reported herein with targeting of DNA vaccines to mucosal surfaces indicates that any antigen can be targeted similarly so long as the means exist to couple the antigen to the M cell ligand.

With regard to the size of the immunogen, the invention again permits a wide range of uses. There is no lower limit brought about by the transporting system according to the invention; thus, any lower limit which might arise would be for reasons specific to the particular intended use or target specificity. It is also possible to convey different immunogens and types of immunogens into the cell at the same time using the conjugates according to the invention.

The ratio of immunogen to M cell specific ligand in the complex or conjugate can vary within a wide range. This ratio will have to be adjusted for each individual case depending on criteria such as the size and structure of the immunogen to be targeted, the size of the linker if required or used, so as to achieve a ratio of transportability and biological activity of the immunogen which is favorable to the particular application.

It is well known in the biological arts that certain amino acid substitutions can be made in protein sequences without affecting the function of the protein. Generally, conservative amino acids are tolerated without affecting protein function. Similar amino acids can be those that are similar in size and/or charge properties, for example, aspartate and glutamate, and isoleucine and valine are both pairs of similar amino acids. Similarity between amino acid pairs has been assessed in the art in a number of ways. For example, Dayhoff et al. (1978) in Atlas of Protein Sequence and Structure, Volume 5, Supplement 3, Chapter 22, pages 345-352, which is incorporated by reference herein, provides frequency tables for amino acid substitutions which can be employed as a measure of amino acid similarity. Dayhoff et al. 's frequency tables are based on comparisons of amino acid sequences for proteins having the same function from a variety of different evolutionary sources. The altered expressed protein may have an altered amino acid sequence, yet still elicits immune responses which react with the antigen protein, and are considered functional equivalents. In addition, fragments of the full length immunogenic proteins may also be constructed. These fragments should comprise an epitope of a protein or peptide which elicits antibodies that cross-react with the immunogenic protein, and are therefore considered to be functional equivalents.

Preparation of Antibodies

Antibodies against M cell ligand-polybasic protein conjugate or other complexes described herein may be prepared by immunizing suitable mammalian hosts using the peptides, polypeptides or proteins alone or conjugated to suitable carriers. It may also be desirable to isolate antibodies to immunogenic epitopes of molecules derived from pathogens, for screening of the phage display minitope libraries described herein. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the hapten. The hapten peptides can be extended at either the amino or carboxy terminus with a Cys residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as antagonists, as well as the intact antibodies. Use of immunologically reactive fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Regions that bind specifically to the desired regions of the gene products can also be produced in the context of chimeras with multiple species origin.

Alternatively, antibodies specific for the M cell ligand polybasic moiety conjugate can be humanized antibodies or human antibodies, as described in U.S. Pat. No. 5,585,089 by Queen et al. See also Riechmann et al., Nature (1988) 332: 323-27.

Use of Phage Display to Identify Immunogenic Epitopes

The immunogen of the M-cell vaccine may be a peptide mimetic, or a minitope, of an infectious agent or a tumor specific antigen, with the nucleotide sequence encoding the minitope being termed a minigene. The method emphasizes the selection of peptide mimetics displayed in a library of modularly coded biomolecules in a phage display format using antibodies or antibody binding domains, and isolation of the nucleotide sequences encoding the same (Burritt et al., Anal. Biochem. (1996) 238:1-13). Display technology has become a routine tool for enriching molecular diversity and producing novel types of proteins orpeptides (Li, Min. Nat. Biotechnol. 12: 1251-1256). The use of display technology circumvents the need for genomic analysis and the difficulty, indeed, the impossibility in some cases, of deducing from gene sequence analyses the structure and antigenic properties of epitopes containing protein, lipid and carbohydrate or formed as a discontinuous sequence of amino acids or monosaccharides. (Scott et al., Science (1990) 249: 386-390).

Regardless of the format, a display library consists of modularly coded molecules, each of which contains three components: displayed entities, a common linker and the corresponding individualized codes. One of the most important characteristics of display technologies is the ability to determine the structure of a desired compound rapidly after initial screening. Structural (or sequence) characterization is often accomplished by a process commonly known as coding and decoding, which can be achieved via a coupled amplification and purification process. There are two requirements for coding strategies: the availability of adequate information space to cover the diverse entities to be displayed and the presence of a highly sensitive and/or amplifiable property that permits rapid decoding. Phage display is one of most commonly used format because of its ease of coding or amplification, but it is limited by the lack of building blocks (only D- and L-amino acids) and structural space for certain desired properties.

The advantage of phage display is recognized in its ability to select peptides that recognize not only conformational epitopes but epitopes consisting of or containing carbohydrates or proteins or conjugates of both (i.e, glycoproteins) (Grobowska et al., Virology (2000) 269: 47-53). It is expected that peptide mimetics for lipid-rich and nucleic acid epitopes may also be selected from a display library with an appropriate antibody probe. For non-biological and some cellular displays, there is virtually no specific limit in terms of what building blocks may be chosen for the generation of diverse molecules, but the coding and decoding strategies are laborious and expensive.

The use of display technology simplifies and reduces the cost of the epitope discovery process for vaccine development because many research reagents, especially antibodies directed against antigenic determinants on pathogens, are already available to select peptide sequences with specific antigenic properties. Because antigenic structures embedded in the cell wall of an infectious agent or capsid of a viral agent have been previously identified by the immune system as immunological targets, a significant amount of guesswork in choosing an effective immunological target is eliminated.

An essential criterion for an antibody to be used for selection of peptides is that it has been demonstrated to bind to specific virulence factors and exert a protective effect in vivo. Monoclonal antibodies are the desired reagent for peptide selection because they bind to a limited number of peptide species. However, in general, polyclonal antibodies are capable of selecting a surprisingly limited number of important prominent antigenic determinants. For example, the selection of sequences from the J404 phage-display library with unfractionated immune serum directed toward filamentous actin in neutrophils yielded only two overlapping consensus sequences (Burritt. et al., J. Biol. Chem. 270: 16974-16980). These two peptides could be traced over the actin crystal structure, which together defined a single epitope. This selection took place in spite of the less dominant antibodies of much broader specificity that would exist in any unfractionated serum. Recovery of epitope mimetics using polyclonal antibodies is therefore driven by the predominantly recognized determinants that are amplified most rapidly from the library. As a practical matter, the use of display technology rapidly and inexpensively defines antigenic domains of complex molecules without having to engage in a sophisticated and expensive genomic discovery process or even to consult gene expression data.

The isolation of putatively immunogenic peptide epitopes bound by protective antibody reagents from phage display peptide libraries may thus aid in preparing the protective vaccines of the invention (Arnon et al., Immunol. (2000) 101:555-562; Wu et al., Gene Therapy (2000) 7:61-69; Qui et al., Hybridoma (1999) 18:103-112;

Kieber-Emmons et al., J. Immunol. (2000) 165:623-627). Because most current efforts in functional proteomics seek to deduce the structure of epitopes from analyses of gene expression products (only proteins), a large and relatively unexplored area of epitope identification is ready for exploitation. Thus, by probing a phage display peptide library with an antibody specific for a pathogen's epitope, a peptide that delineates or mimics a continuous or, more importantly, a discontinuous or conformational epitope—an antibody binding determinant composed of residues distant in the primary sequence but adjacent in the folded protein structure—can be isolated.

Because the oligonucleotide sequence encoding a given peptide mimetic is derived from the sequence analysis of phage DNA, minigenes for epitope expression in vivo can be readily prepared for use in DNA-based vaccine development, or for use in fusion protein vaccines, or to recombinantly produce peptide epitopes to be complexed to vaccines, from stocks of phage identified as expressing relevant mimetics. Minigene expression (of immunogenic peptides) facilitated by promoter genes of eucaryotic cells can yield protective immune responses following vaccine delivery or targeting to compartments of the immune system.

Because the antigenicity of the phage-derived peptide epitopes is immediately established (it was, after all, selected by an antibody) and their immunogenicity can be assessed by immunizing animals with appropriate carrier formulations (and by other techniques known to persons skilled in the art), the minigene encoding the peptide can predictably be expected to elicit immune responses if the peptide expressed in vivo appropriately conforms to the structure of the native epitope. Minigenes, comprised for example of 27 to 45 nucleotide base pairs, and encoding nine-mer to fifteen-mer minitopes can be incorporated into plasmid constructs and are able to elicit protective immune responses to an infectious agents. Biological display exploits the cellular biosynthesis machinery to assemble biopolymers, the sequence of which ultimately specifies structure and distinct properties.

Phage display formats are most commonly employed where the coding sequence is embedded in the viral genome and the displayed molecule is part of the viral coat protein. In utilizing phage display techniques as described herein, the peptide epitopes preferably are displayed as sequences of nine amino acids as an amino-terminal fusion with the minor coat protein pIII. These are “linked” to codes that have chemical and physical properties that can be readily determined (e.g. the sequence of nucleic acids).

Minigenes or minitopes are predicted to have significant advantages as subunit vaccine candidates when compared to conventional vaccine components. In particular, it is the ability of antigen DNA encoding epitopes or peptide mimetics of epitopes, given the proper delivery system, to elicit T-dependent immune responses that is advantageous (Cox et al., Science (1994) 254: 716-719; Celis et al., Proc. Natl. Acad. Sci. (1994) 91: 2105-2109; Wang et al. Science. (1998) 282: 476-480). Peptides can induce Th1 responses (CTL, IgG2a. DH etc.) when minigenes encoding the peptide and appropriate eucaryotic expression or promoter genes (Th, CMV) are incorporated into DNA plasmids for vaccination (Grobowska et al., Virology (2000) 269: 47-53; Kawabata et al., Infect. Immun. (1999) 67: 5863-5869). Several studies have shown that a cell-mediated immunity is an important feature of the host's response to peptide or DNA vaccines. (Shikhman. et al., Nat. Biotech. (1997) 15: 512-516; Ulmer. et al., Science (1993) 259: 1745-1749; Kieber-Emmons. et al., J. Immunol. (2000) 165: 623-627, Kawabata et al., Infect. Immun. (1999) 67:5863-5869; Van Ginkel et al., Emerg. Infect. Dis. (2000) 6(2): 123-132; Ghose et al., Mol. Immunol., 25(3): 223-230).

Peptides that mimic carbohydrate epitopes also have significant advantages as vaccines compared with carbohydrate conjugate vaccines. (Qui et al., Hybridoma (1999) 18:103-112; Kieber-Emmons et al., J. Immunol. (2000) 165:623-627 Pincus. et al., J. Immunol. (1998) 160: 293-298; Phalipon. et al., Eur. J. Immunol. 10: 2621-2625). Carbohydrate antigens are classed as T cell-independent antigens which gives them immunological properties much different from those associated with protein antigens. Because pure carbohydrate vaccines provoke narrowly defined immune responses, predominantly serum IgM, their use in vaccines have limited effectiveness in combating disease. Conjugate vaccine technology has overcome some of the limitations of carbohydrate antigens because of the T-dependent help conferred by the carrier protein. However, carbohydrate conjugate vaccines induce immune responses that are deficient in many respects, including the lack of induction of the Th1-associated IgG2a isotype and cell-mediated immune responses to pathogens and tumor cells.

The immunogenicity of carbohydrate and conformational minitopes associated with bacterial and fungal pathogens has been demonstrated by the inventors. For example, it has been demonstrated that minitopes of carbohydrate antigens of Candida albicans and group B streptococci are both antigenic and immunogenic when administered as conjugates or, as in the case of group B streptococcoal minitopes, as purified peptides.

Vaccine Formulation

The amount of expressible DNA or transcribed RNA to be introduced into a vaccine recipient will have a very broad dosage range and may depend on the strength of the transcriptional and translational promoters used as well as subject size, e.g., human versus bison (i.e., in bison, 5 mg of DNA can be an effective dose). In addition, the magnitude of the immune response may depend on the level of protein expression and on the immunogenicity of the expressed gene product. In general, effective dose ranges of about 1 ng to 5 mg, 100 ng to 2.5 mg, 1 μg to 750 μg, and preferably about 10 μg to 300 μg of DNA is administered intranasally. It is also contemplated that booster vaccinations may be provided. Following vaccination with M cell ligand-polybasic conjugate-polynucleotide complexes, boosting with the encoded antigen products is also contemplated. Parenteral administration, such as intravenous, intramuscular, subcutaneous or other means of administration of interleukin-12 protein (or other cytokines, e.g. GM-CSF), concurrently with or subsequent to intranasal introduction of the M cell ligand-polybasic conjugate-polynucleotide complex of this invention may be advantageous.

The polynucleotide and other immunogens of the invention may be associated with adjuvants or other agents which affect the recipient's immune system. In this case, it is desirable for the formulation to be in a physiologically acceptable solution, such as, but not limited to, sterile saline or sterile buffered saline. The active immunogenic ingredients can be mixed with excipients or carriers which are pharmaceutically acceptable and compatible with the active ingredient. Suitable excipients include but are not limited to water, saline, dextrose, glycerol, ethanol, or the like and combinations thereof.

In addition, if desired, the DNA and other vaccine complexes may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-( 1′-2′-dipalmitoyl-sn-glycero-3-hydroxyphosphoryloxy)-ethylamine (CGP 19835A, referred to as MTP-PE); and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogen resulting from administration of the immunogen in vaccines which are also comprised of the various adjuvants. Such additional formulations and modes of administration are known in the art and can also be used.

The DNA and other vaccines of the present invention may be formulated into compositions as neutral or salt forms. Pharmaceutically acceptable salts include but are not limited to the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

The M cell ligand-polybasic moiety (or conjugate)-polynucleotide compositions and other vaccine compositions of the present invention are administered in a manner compatible with the dosage formulation, and in such amount as will be prophylactically and/or therapeutically effective. The quantity to be administered, which is generally in the range of about 100 to 1,000 μg of protein per dose, more generally in the range of about 5 to 500 μg of protein per dose, depends on the subject to be treated, the capacity of the individual's immune system to synthesize antibodies, and the degree of protection desired. Precise amounts of the active ingredient required to be administered may depend on the judgment of the physician and may be peculiar to each individual, but such a determination is within the skill of such a practitioner.

The DNA and other vaccines of the present invention may be given in a single dose or multiple dose schedule. A multiple dose schedule is one in which a primary course of vaccination may include 1 to 10 or more separate doses, followed by other doses administered at subsequent time intervals as required to maintain and or reinforce the immune response, e.g., at 1 to 4 months for a second dose, and if needed, a subsequent dose(s) after several months.

Immunization by DNA injection allows the ready assembly of multicomponent subunit vaccines. Simultaneous immunization with multiple influenza genes has recently been reported. (Donnelly et al., Vaccines (1994) pp 55-59). The inclusion in a DNA vaccine of genes whose products activate different arms of the immune system may also provide thorough protection from subsequent challenge.

The vaccines of the present invention are useful for administration to domesticated or agricultural animals, as well as humans. Vaccines of the present invention may be used to prevent and/or combat infection of any agricultural animals. The techniques for administering these vaccines to animals and humans are known to those skilled in the veterinary and human health fields, respectively.

Except as may be noted hereafter, contemplated techniques for cloning, DNA isolation, amplification and purification, for enzymatic reactions involving DNA ligase, DNA polymerase, restriction endonucleases and the like, and various separation techniques are those well known and commonly employed by those skilled in the art. A number of standard techniques are described in Sambrook et al. (1989) Molecular Cloning: A Laboratory Manual, Second Edition, Cold Spring Harbor Laboratory, Plainview, N.Y.; Maniatis et al. (1982) Molecular Cloning: A Laboratory Manual, Cold Spring Harbor Laboratory, Plainview, N.Y.; Wu (ed.) (1993) Meth Enzymol. 218, Part I; Wu (ed.) (1979) Meth Enzymol 68; Wu et al. (eds.) (1983) Meth Enzymol 100 and 101; Grossman et al. (eds.) Meth Enzymol 65; Miller (ed.) (1972) Experiments in Molecular Genetics, Cold Spring Harbor Laboratory, Cold Spring Harbor, N.Y.; Old et al. (1981) Principles of Gene Manipulation, University of California Press, Berkeley; Schleif et al. (1982) Practical Methods in Molecular Biology; Glover (ed.) (1985) DNA Cloning Vol. I and II, IRL Press, Oxford, UK; Hames et al. (eds.) (1985) Nucleic Acid Hybridization, IRL Press, Oxford, UK; Setlow et al. (1979) Genetic Engineering: Principles and Methods, Vols. 1-4, Plenum Press, New York. Abbreviations and nomenclature, where employed, are deemed standard in the field and commonly used in professional journals such as those cited herein.

The following examples are provided to illustrate the present invention without, however, limiting the same thereto.

### EXAMPLES

### Example 1

Production of Recombinant Reovirus σ1 Protein

The cloned protein σ1 cDNA from reovirus serotype 3 strain in the pST3-S1 Banjedjea et al., Virology (1988) 167:601-612) was kindly provided by Dr. Wolfgang K. Jolik from Duke University Medical Center. For its expression in E. coli, using PCR, a 1.4 kb cDNA fragment containing the restriction endonuclease sites, EcoR 1 and Pst 1, was inserted into the polylinker site of an E. coli expression plasmid, pMAL-C2 (New England Biolabs, Beverly, Mass.). The resultant, pMAL-C2-S1, was used to transform E. coli, strain BL21 (DE3; Novagen, Madison, Wis.). Upon induction with IPTG, the maltose-binding protein (MBP)::protein σ1 fusion protein was induced in the cytoplasm of E. coli. The clear lysate of E. coli containing the fusion protein was purified by affinity chromatography using amylose resin according to manufacturer's directions (New England Biolabs). This MBP::protein σ1 fusion protein is referred to as recombinant protein σ1.

### Example 2

Preparation of Recombinant Fusion Protein σ1-polylysine-DNA Complex

The recombinant protein σ1 was covalently linked to poly-L-lysine (PL) according to the methods of Wagner et al (1990). Protein σ1 was purified and resuspended in phosphate-buffered saline (PBS), pH 7.3. To generate the dithiopyridine linker, both protein σ1 and PL were each modified with succinimidyl 3-(2-pyridyldithio)propionate (SPDP; Sigma Chemical Co., St. Louis, Mo.). Briefly, in separate vessels, ten milligrams of protein σ1 in 5 ml PBS, pH 7.3, and twenty milligrams of PL (Sigma), with an average chain length of 270 lysine monomers, in 1 ml of 75 mM sodium acetate were each vigorously mixed to react with SPDP in 15 mM ethanolic solution for one hour. The resulting SPDP modified protein σ1 was then dialyzed against PBS, pH 7.3, and the modified PL was then dialyzed against 20 mM sodium acetate to remove unbound SPDP. To generate the mercaptopropionate linker, the resultant PL with dithiopyridine linker was further mixed with 23 mg dithiothreitol (DTT) in sodium bicarbonate solution, pH 7.5, for one hour under argon. The mercaptopropionate PL was dialyzed against 20 mM sodium acetate to remove free DTT. The 10 mg of dithiopyridine-modified protein σ1 was then mixed with the 20 mg of mercaptopropionate-modified PL under argon at room temperature for 18 hours. The resultant reaction generated what is referred to as protein σ1-PL conjugate. This conjugate was dialyzed to remove unreacted mercaptopropionate-PL using a membrane with an exclusion of 100 kilodaltons, against HEPES buffered saline (20 mM HEPES, 100 mM sodium chloride, pH 7.4; HS). Protein σ1-PL concentration was determined using a Bradford assay (Pierce, Rockford, Ill.). For control transfections, MBP-PL conjugates were similarly generated. For the formation of conjugate-DNA complex, the protein σ1-PL conjugate in 125 μl of HS was added dropwise into an equal volume of HS containing the plasmid DNA and incubated at room temperature for 30 minutes to form conjugate-DNA complex.

### Example 3

Cell Ligand Binding Assay

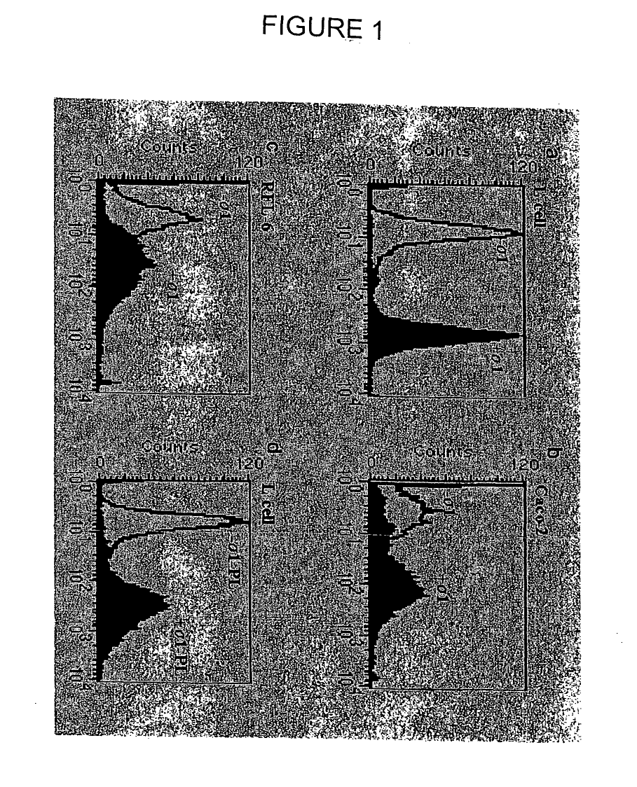

To assess the cell-binding capacity of the protein σ1 and protein σ1-PL conjugates, an immunofluorescent binding assay was performed. The protein σ1 and σ1-PL conjugates were incubated with mouse L cells (CCL-1, American Type Culture Collection, Manassas, Va.), RFL-6 fibroblast cells (CCL-192, ATCC), and Caco-2 cells (HTB-37, ATCC) and binding was assessed using 20 μg/ml of biotinylated monoclonal anti-reovirus protein σ1 antibody (HB-167, ATCC) and SA-PE (Southern Biotech. Assoc., Birmingham, Ala.), and specific binding was then assessed using flow cytometry. Protein σ1 was able to bind to all three cell types (FIG. 1). No staining was obtained with normal rabbit serum or in the presence of SA-PE only.

### Example 4

Cell Culture and Transfection with Plasmid DNA

The mouse L cells, RFL-6 cells, and Caco-2 cells were used for targeting gene transfer by protein σ1-PL conjugate. The mouse L cells have been used as the in vitro model for reovirus protein σ1 binding studies. Cells were maintained in complete media: Dulbecco's minimum essential medium (DMEM; BioWhittaker, Walkersville, Md.), supplemented with 10% fetal bovine serum (Life Technologies, Grand Island, N.Y.) at 37° C. under 5% CO₂.

For Luc assay, 2.5×10⁵ cells were added to each well of the 12-well plate and allowed to adhere overnight. The conjugate-DNA complexes were added and incubated for another 24 hours in complete media. For chloroquine treatment, the cells were incubated with protein σ1-PL-DNA complexes and 100 μM chloroquine for 4 hours at 37° C. Four hours after incubation, the conjugate-DNA complexes were removed, and cells were incubated with complete media for another 24 hours. The cells were lysed to assay reporter gene activity.

For β-Gal assay, 5×10⁵ cells were added to each well of 6-well plate and allowed to adhere overnight. The conjugate-DNA complexes containing 8 μg ρ1-PL and pCMVβ-gal (Life Technologies), with or without chloroquine, were added and incubated for 24 hours. The cells were then incubated with fresh media for another 24 hours prior to flow cytometry analysis.

### Example 5

Assays for Reporter Gene Detection

Luciferase

The Luc gene was used as a reporter gene to assay protein σ1-PL conjugate-mediated transfection. A 1.4 kb Luc gene fragment flanked with Hind III and EcoR V was extracted from pSPKuci(+) (Promega, Madison, Wis.). The pCMVLuciferase (pCMVLuc) was constructed by ligating the 1.4 kb luciferase gene into the polylinker site in pcDNA3.1(+) (Invitrogen, Carlsbad, Calif.). L cells were exposed to 2 μg pCMVLuc complexed with 8 μg σ1-PL and 24 hours later cells were assessed for expression of Luc by lysing the cells with 1× luciferase lysis buffer (Promega, Madison, Wis.), and mixing twenty μl of supernatant of cell lysates with 100 μl of Luc assay buffer. Luc levels were quantitated with a luminometer (LUMAT LB 9507, EG&G Berthold, Germany). The relative light units from the total lysates were used to express the Luc activities produced from each transfection.

Luc activity (486940±43954) could be detected in several independent experiments as a consequence of σ1-PL-pCMVLuc transfection. Significant expression of Luc was also achieved with RFL-6 and Caco-2 cells, of which Luc activities were 40684±6633 (n=6) and 40703±6225 (n=6), respectively. The transfection did appear to be mediated by protein 1 when compared to the various control transfections. The background activity of Luc is 193±29 (n=6). Minimal detectable Luc activity could be measured when L cells were transfected with pCMVLuc only, protein σ1 associated, but not covalently attached with PL-pCMVLuc, or PL complexed with pCMVLuc without any protein σ1. Thus, optimal transfection required the covalent attachment of protein σ1 for optimal cellular transfection.

To show specificity of protein σ1-mediated gene transfer, transfection of L cells was performed using protein σ1-PL-pCMVLuc in the presence of excess, unconjugated recombinant protein σ1 or in the presence of an antireovirus polyclonal antibody. If indeed, the cell transfection was receptor-mediated, these molecules should inhibit transfection. As such, the presence of increasing concentrations of unconjugated protein σ1 resulted in the attenuation of gene transfer with an IC₅₀ of approximately 75 μg/ml for protein σ1. Between 100 and 500 μg/ml of uncomplexed protein σ1 resulted in nearly 100% inhibition of cell transfection.

As for Caco-2 cells, 100 and 500 μg/ml of uncomplexed protein c1 caused a respective 89% and 94% inhibition of Luc expression (data not shown). Protein σ1-mediated transfection was receptor-specific since bovine serum albumin (BSA) could not inhibit protein σ1-PL-mediated gene transfer. Gene expression in the presence of BSA at 0.5 mg/ml was 899000±145720 light units (n=3).

As with the recombinant protein σ1, the polyclonal anti-reovirus 3 antibody showed inhibition of the protein σ1-PL-pCMVLuc gene transfer to mouse L cells in a dose-dependent fashion. Maximal inhibition was obtained with 10-fold diluted anti-reovirus 3 antibody resulting in a 98% decrease in Luc gene expression. Normal rabbit IgG did not inhibit protein σ1-PL-mediated gene transfer. Interestingly, dilute anti-reovirus 3 antibody (1:100) appeared to slightly enhance transfection with this DNA complex, possibly due to a prozone effect from antibody and protein σ1 concentrations. Collectively, these findings demonstrate that protein σ1-PL mediated gene transfer is accomplished via ligand binding to target cells.

β-Galactosidase

Expression of β-Gal was visualized by incubating the transfected cells with PBS solution containing 1 mg/ml of 5-boromo-4-chloro-3-indolyl-β-galactopyranoside (X-Gal, Boeringer Mannheim, Indianapolis, Ind.) at 37° C. for 16 hr. To quantify the transfection efficiency, cells having been transfected with the constructs PL-pCMV-β-Gal (Life Technologies) or protein σ1-PL-pCMV-β-Gal were harvested, loaded with 200 μM fluorescein-mono-β-D-galactopyranoside (FDG; Molecular Probe, Eugene, Oreg.) for 30 minutes at 37° C. and diluted with cold PBS to a final concentration of 2.5×10⁵ cells/ml. Flow cytometry analysis was performed using a Becton Dickinson FACSCalibur.

Transfection with protein σ1-PL-pCMV-β-Gal complexes produced a statistically significant higher level of β-Gal expression than that observed with protein PL-pCMV-β-Gal in both L cells and RFL-6 cells. Preliminary staining showed that Caco-2 cells exhibit endogenous β-Gal activity, therefore, these cells were not evaluated further. Transfection with protein PL-pCMV-β-Gal (lacking protein σ1) was ineffective and levels of β-Gal expression did not differ from background levels.

### Example 6

Histochemical Determination of Fusion Protein (σ1 Binding to NALT

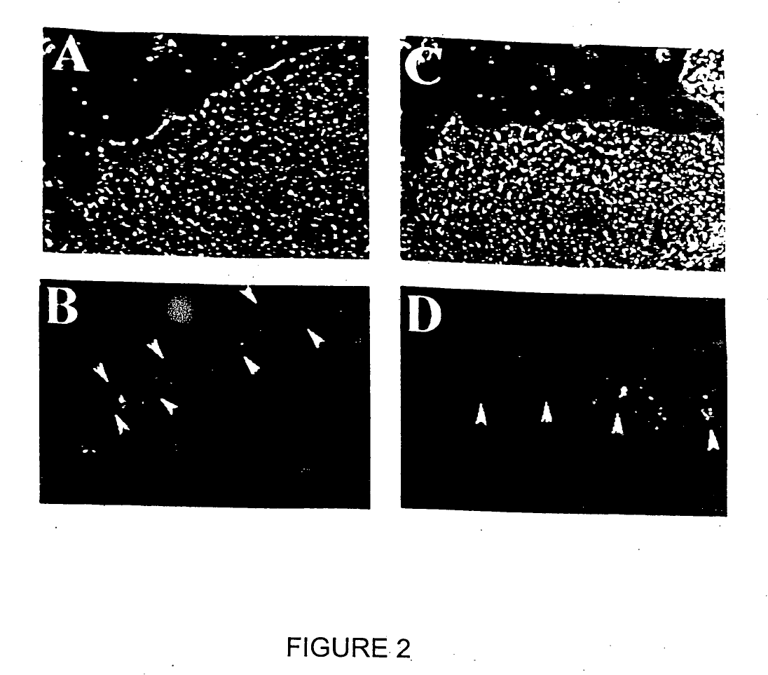

NALT tissues were collected as previously described (Asanuma et al., J Immunol Methods (1997) 202:123-131 and Heritage et al., Am J Respir Crit Care Med (1997) 156(4 Pt 1): 1256-1262). Palates with visible NALT were washed in DMEM, and prior to binding with biotinylated protein σ1 (following standard procedures), NALT were first incubated in DMEM alone or in the presence of 500 μg/ml of protein σ1 in DMEM with gentle rotation on a GeneMate orbital Shaker (Intermountain Scientific Co., Bountiful, Utah) for 45 minutes at 4° C. Recombinant protein σ1 was able to bind to NALT. See FIG. 2. Control sections, incubated with SA-Horseradish peroxidase (HRP) only, failed to stain. NALT were incubated with excess unmodified protein σ1 in order to inhibit biotinylated protein σ1 binding, and thus, show specificity of binding to the NALT.

NALT were then washed gently in DMEM and incubated in 50 μg/ml biotinylated protein σ1 in DMEM, and were again rotated gently for 45 min at 4° C. Following incubation, NALT were removed, rinsed gently in PBS, and then arranged in 15 mm by 15 mm Tissue Tek® Cryomold (Miles Inc., Elkhard, Ind.) with their ventral surfaces oriented toward the bottom of the mold. The palates were then frozen in Tissue Tek® O.C.T. compound embedding media and stored at −80° C. until use. For immunoperoxidase staining, frozen NALT sections, previously treated with biotinylated protein σ1, were cut at 5 mm, air dried, fixed in acetone at 4° C., and air dried before rehydration.

Frozen sections were rehydrated in Dulbecco's PBS (DPBS) containing 0.2% normal goat serum (NGS). A 1:250 dilution of SA-HRP conjugate (BioSource International, Camarillo, Calif.) was added for 45 min at room temperature. The location of the HRP was visualized upon reaction with the precipitable substrate, 3-amino-ethylcarbazole (AEC: Sigma). Results showed that binding of biotinylated fusion protein σ1 was competitively inhibited by excess unlabeled fusion protein σ1.

### Example 7

In Vivo Analysis of Intranasal Immunization with GI Conjugates:

Luciferase

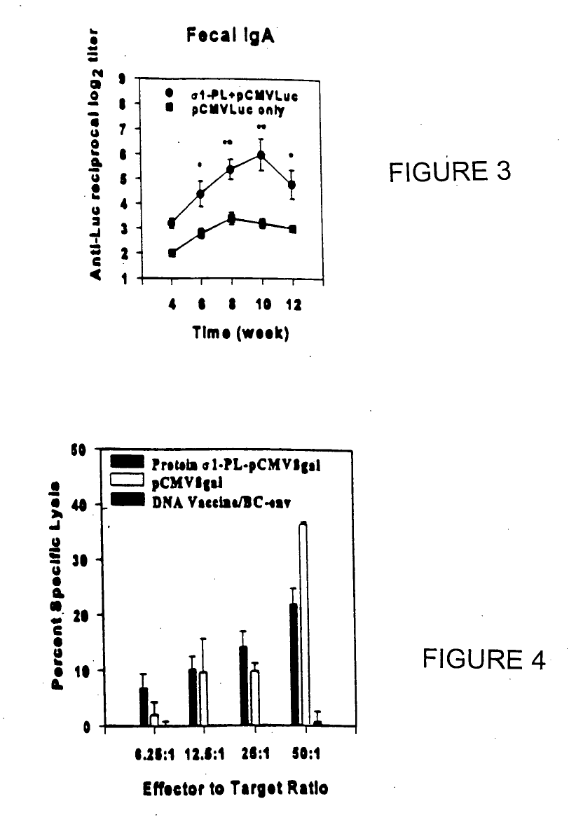

Intranasal (i.n.) immunization with protein σ1-polylysine (PL) conjugate enhances induced mucosal IgA responses in mice. Data depicts the mean endpoint titers (±SE) for mice immunized i.n. with protein σ1-PL-pCMVLuciferase (Luc) or uncomplexed pCMVLuc (5 mice/group). Significant differences between protein σ1-PL-pCMVLuc and pCMVLuc only were determined by student t-test. *p<0.05.**p<0.005. (See FIG. 3).

### Example 8

β-Galactosidase

Intranasal (i.n.) immunization with protein σ1-PL-pCMVβ-galactosidase (βgal) stimulates βgal-specific CTL responses in mice. BALB/c mice received three i.n. immunizations with either protein β1-PL-pCMVβgal or pCMVβgal. Immune splenocytes were able to lyse ⁵¹Cr loaded βgal-expressing fibroblasts (BC-βgal), but not irrelevant BC-envelope (BC-env) targets. The mucosally formulated DNA was as efficient in stimulating βgal-specific CTLs as those mice receiving naked DNA. (See FIG. 4).

### Example 9

HIV

Intranasal (i.n.) immunization with protein σ1-polylysine (PL) conjugate was conducted to enhance induced mucosal IgA responses in mice. The mean endpoint titers (±SE) for mice immunized i.n. with protein σ1-PL-pCMVgp160 and σ1-PL-pCMVgp140 or uncomplexed pCMVgp160 and pCMVgp140 (5 mice/group) was compared. Significant differences between protein σ1-PL-pCMVgp160 and σ1-PL-pCMVgp140 versus pCMVgp160 and pCMVgp140 only were determined by student t-test. Using the mucosal DNA formulation, the same magnitude of IgG antibody response is observed as was observed for the anti-reporter gene responses.

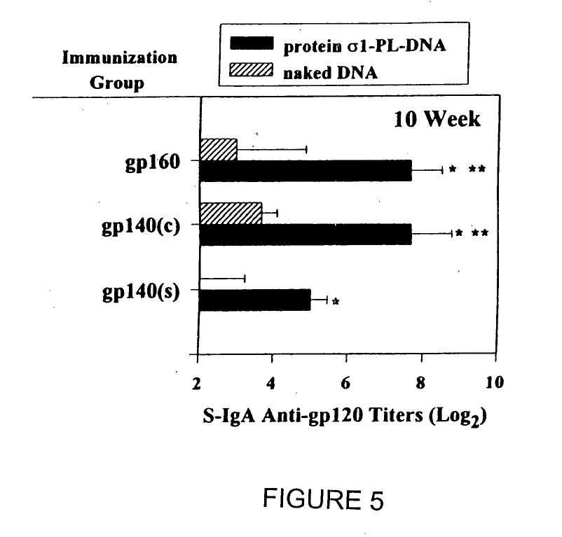

Experimentally, mice were immunized with one of three designated HIV DNA vaccine constructs, that is gp160, gp140(c) and gp 140(s), as indicated in FIG. 5. Each group (5 mice/group) received three intranasal immunizations either of naked DNA or of the identified M cell DNA vaccine formulation. As indicated, the mucosal intestinal IgA response was elevated 10 weeks after the initial immunization when compared to intranasal naked DNA immunization. Thus, the DNA vaccine formulation improved mucosal IgA responses when compared to conventional naked DNA immunization.

### Example 10

Brucella

Intranasal (i.n.) immunization with protein σ1-polylysine (PL) conjugate enhances induced mucosal IgA and IgG responses in bison. The mean endpoint titers (±SE) for bison immunized i.n. with protein σ1-PL-pCMVL7/L12 ribosomal protein or uncomplexed pCMVL7/L12 ribosomal protein (5 bison/group) was compared. Significant differences between protein σ1-PL-pCMVL7/L12 ribosomal protein versus pCMVL7/L12 ribosomal protein only were determined by student t-test. Using our mucosal DNA formulation, we observed increases in serum IgG and vaginal IgA and IgG anti-L7/L12 antibody titer in bison.

### Example 11

HIV gp120

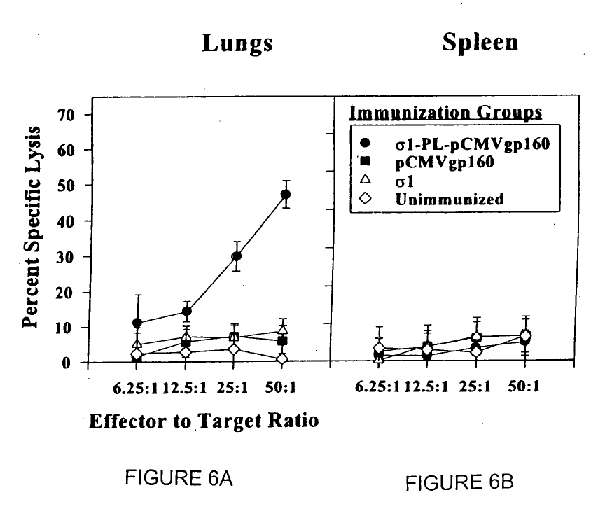

Intranasal immunization with an M cell-formulated HIV DNA vaccine promotes enhanced cytolytic activity (cell-mediated immunity) against target cells expressing HIV gp120 as shown in FIGS. 6A and 6B. Mice received a formulated vaccine, naked DNA version, protein sigmal by the intranasal route three times at one week intervals or were left unimmunized. Mice were sacrificed six weeks subsequent to this initial immunization to procure specified tissues. In a dose-dependent fashion, the lungs from only mice receiving only the formulated vaccine showed effector function. These results show that the vaccine as formulated is superior to naked DNA in stimulating gp120-specific immunity.

Data also indicated that antigen restimulation specifically enhances CTL responses from mice i.n.-immunized with the formulated vaccine as opposed to mice immunized with the naked DNA alone. Pulmonary lymph nodes (LRLN) and splenocytes from immunized mice were restimulated in vitro with cells expressing gp120 or beta-galactosidase (neg. control), and were subsequently examined for cytolytic activity. The observed killing was specific since negative targets were not lysed, and other mechanisms of vaccination failed to stimulate cytolytic activity.

### Example 12

Method for Selection of Antigenic Peptide and Minigene

The J404 nonapeptide library was employed and expressed on the surface of a new vector M13KBst (1) for defining polynucleotide sequences and the nonapeptides they encode. The library has a complexity of 5×10⁸ unique phage. The display technology has been refined and is practiced as described below. Briefly, affinity purification of phage bearing epitopes bound by antibody reagents are performed as follows: 1×10¹² phage (75 μl ) from the nonapeptide J404 library are combined with 300 μl of Sepharose beads conjugated with 1.50 mg of the monoclonal reagent or polyclonal antiserum. Alternatively, if the selecting antibody is an IgG, protein A or G or anti-IgG-coated, sepharose beads may be coated with selecting antibody for the interaction with phage. The beads are then mixed with the phage at 4° C. for 16 hours by gentle rotation. The mixture is then loaded into a 5 ml plastic column barrel (Evergreen) and unbound phage removed by washing with 50 ml phage buffer (50 mM Tris-Cl pH 7.5, 150 mM NaCl, 0.5% Tween 20 (v/v), 1 mg/ml BSA). Bound phage are eluted from the column with 2.0 ml of eluting buffer (0.1 M glycine, pH 2.2), and the pH of the eluate neutralized immediately with four drops of 2 M Trizma base. The titer of the eluate phage is determined for each column eluate by plaque assay according to standard procedures.

The column matrices are preserved for reuse in second and third round affinity purifications by washing with 10 ml PBS pH 7.0, followed by 3.0 ml PBS containing 0.02% sodium azide. The column is stored at 4° C. until the next affinity purification, and prepared for reuse by rinsing with 20 ml of phage buffer prior to mixing with amplified phage.

In order to select strongly binding sequences from the library, three rounds of selection, alternated with two rounds of amplification are carried out as has been previously described in Burritt. et al., Anal. Biochem. (1996) 238: 1-13; Burritt. et al., J. Biol. Chem. (1995) 16974-16980. Utilization of one or two of these sequences requires identification of those most strongly bound by the mAb. Typically, this is done with a plaque lift to select clones from the third round eluate that give strong signals with the selecting antibody. Individual clones are picked and amplified and confirmation of specific activity can then b e done by ELISA. Specifically, phage at 10⁹ to 10¹¹ plaque forming units are exposed to wells of an ELISA plate which had been coated with 75 μl of antibody (mAb-114 or KPL polyclonal, for example), diluted to 200 mg/ml in PBS, pH 7.4. After incubation of the phage pool on the antibody, the wells are washed and then probed with a rabbit anti-phage polyclonal antibody (1:20,000 dilution in PBS) for one hour at 25° C. Following additional washing, the wells are exposed to goat anti-rabbit polyclonal secondary antibody tagged with HRP for one hour at 25° C. After a final wash, the color is developed with a standard o-phenylenediamine in citrate buffer system and absorbances recorded at 490 nm.

Peptide sequences can be determined by sequencing of the N-terminal chimeric pIII-peptide gene using automated sequencing methodology or using techniques which are well-known in the art. For reference in this regard, see Current Protocols in Molecular Biology, Wiley Interscience, 1989. These analyses yield the polynucleotide (minigene) sequence used for preparing synthetic oligonucleotide minigenes for ligation to plasmid DNA. Minigenes encoding peptides with greatest binding properties for the antibody probe are selected for vaccine development.

### Example 13

Preparation of Minitopes and Minigenes for Candida vaccine Development

Infections with the pathogenic fungus, Candida albicans, are represented by disseminated as well as mucocutaneous forms of candidiasis. In disseminated forms of candidiasis, the organism infects many tissues and organs often with fatal outcomes especially in patients with impaired immune systems. As part of the normal flora of the skin, intestine, nose and throat, the organism may produce mucocutaneous disease such as oral candidiasis (thrush) and candidal vaginitis. There are no effective vaccines available for human use. Studies (Knabe et al., Infect. Immun. (1994) 62: 1662-1668) have identified and characterized a protective epitope chemically defined as β-1,2 trimannose which is embedded in the cell wall of C. albicans. Cell wall fractions containing a phosphomannan complex (PMC) or purified trimannose preparations administered with adjuvants or on liposomes elicit antibody responses and protect mice against challenge with C. albicans.

A family of peptides recognized by the monoclonal antibody MAb B6.1, an antibody of the IgM that binds to PMC or β-1,2-trimannose, has been previously identified. Mab B6.1 was obtained from mice immunized with a phosphomannan protein complex (PMC) which contains an adhesin domain used by Candida for attachment to target cells of the host. B6.1 is specific for a β-1,2-trimannose carbohydrate moiety that is phosphodiester within the other mannan complexes expressed on or near the surface of yeast cells.

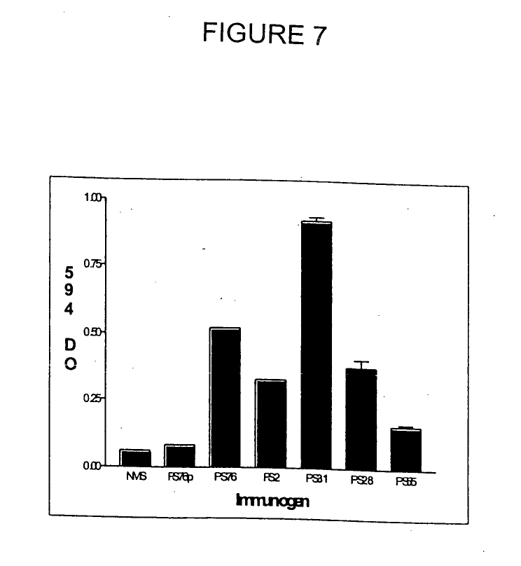

Mab B6.1 protects against disseminated and mucocutaneous candidiasis in murine models of infection and has been shown to react with a cell wall of Candida albicans. Probing the J404 phage display library with MAb B6.1 selected five peptides rich in aromatic and hydroxylated amino acids with a consensus of Ar—X—X—Ar-Z-Z-Z-Ar—Ar (Ar═W, F, or Y; X=any amino acid; and Z═S, T, or preferentially G). Each of these peptides is nine amino acids in length and they bind preferentially to B6.1. Both phage-displayed peptides and synthetic peptide-carrier protein conjugates showed specific reactivity with MAb B6.1. FIG. 7 demonstrates that the Candida carbohydrate epitope demonstrates dose dependent inhibition of antibody binding to all five phage-displayed minitopes and to a synthetic peptide-carrier protein conjugate. Some of the phage-displayed minitopes (PS76, PS2, and PS31) demonstrated dose-dependent inhibition of antibody binding to the B6.1 carbohydrate epitope. Balb/c mice were immunized subcutaneously with phage-displayed peptides (clone numbers PS76, PS2, PS31 PS28 and PS55), synthetic peptide (PS76p), with Ribi RS-700 adjuvant and boosted at day 21 and 35. Immune serum samples were screened by agglutination against C. albicans cells and by ELISA against carbohydrate extracts with enzyme conjugated secondary antibody to detect binding of mouse IgG plus IgM. The results shown in FIG. 7 indicate that the selected peptide mimetics act antigenically and immunologically.

A Candida minigene as described above was constructed with the polynucleotide sequence, 5′-TAT,CGT,CAG,TTT,GTG,ACG,GGT,TTT,TGG-3′ (SEQ ID NO: 1), encoding the PS76 peptide (YRQFVTGFW) (SEQ ID NO: 2) for incorporation into a DNA vaccine construct. The minigene is incorporated into a plasmid as described above and administered to a mammalian host in order to elicit an immune response. Such vaccine constructs also include mammalian promoters controlling transcription, preferably the CMV promoter, a T cell epitope and a mucosal adjuvant preferably cholera toxin (CT) to stimulate both arms (Th1 and Th2) of the immune system.

### Example 14

Preparation of Minitopes and Minisenes for Group B Streptooccal Vaccine Development

Group B streptococci (GBS) are a major cause of neonatal sepsis and meningitis. GBS are one of many examples of microbial polysaccharides that are notably poor immunogens. Efforts to prevent this disease using GBS carbohydrates are marginally effective in eliciting antibody or a protective immune response. Immunogenic minitopes of the type III capsular carbohydrate of group B Streptococcus (GBS) have been developed (Pincus. et al., J. Immunol. (1998) 160: 293-298). The murine mAb S9, a protective antibody against the type III capsular polysaccharide of group B streptococci, was used to select epitope analogues from the J404 peptide display phage library. Two populations of phage were identified with displayed sequences of WENWMMGNA (SEQ ID NO: 3) and FDTGAFDPDWPA (SEQ ID NO: 4). The binding of anti-GBS antibody to GBS was inhibited by the free peptide and significant antibody responses to GBS and purified capsular polysaccharide was elicited by a single immunization with peptide mimetics Moreover, a single dose of peptide mimetic induced a greater anti-GBS antibody response than that seen following infection with 108 colony forming units of GBS.

M cell:DNA vaccines for GBS have been prepared by ligating the polynucleotide sequence 5′-TTT,GAT,ACG,CTG,GCT,TTT,GAT,CCT,GAT,TGG,CCT,GCT-3′ (SEQ ID NO: 5) encoding peptide FDTGAFDPDWPA (SEQ ID NO: 4) into either plasmid pcDNA3.1 (Invitrogen, Carlsbad, Calif.) or pCMV-SPORT-bgal (GIBCO) under a CMV promoter. To assess the efficacy of vaccine constructs, adult BALB/c mice immunized immunized intranasally with three doses of M cell vaccine have been shown to produce high titers of antibody against GBS.

### Example 15

Preparation of Minitopes and Minigenes for Brucella a Vaccine Development

Numerous studies have clearly demonstrated that protective immunity against brucellosis correlates with antibodies administered passively or elicited by immunization against the 0-antigenic domain of S-LPS (Cloeckaert, A. et al., Infect. Immun. (1992) 60: 312-315; Dubray, G., Annales de l'Institut Pasteur. Microbiologie (1987) 138: 84-87).

The lipopolysaccharide of smooth Brucella (S-LPS) has been found to contain two distinct epitopes designated A and M (Corbel, M., 1st Int. Conf. Emerg. Zoonosis. (1997) 3: 213-221). The relative amounts of the two epitopes vary among smooth Brucella strrains, and these epitopes are absent on rough strains. The S-LPS structure has been defined as homopolymers of 4,6-dideoxy-4-formamido-a-D-mannopyranose residues. The A antigen contained in S-LPS is a linear α-1,2-linked polymer with about 2% β-1,3-linkages, while the M antigen is a linear polymer of pentasaccharide repeating units containing one α-1,3-linked and four α-1,2-linked monosaccharide residues. Apparently, the α-1,3-linkage is the major part of the structure recognized by anti-M mAbs in S-LPS of B. abortus, B. melitensis, and B. suis which express the M epitope in variable amounts. The structure of the common epitope is unknown. The J404 phage library has been probed with an anti-A antibody, YsT9-1 (a IgG1 mAb obtained from the Canadian Research Council) to identify peptide mimetics with high conformational similarity to the α-1,2 linked carbohydrate residues peculiar to the A antigen. Minitope and minigene vaccine candidates were prepared as previously described.

Probing the J404 phage display library with mAb YsT9-1 which binds to B. abortus A antigen yielded a nonapeptide consensus sequence of VSWCSSCSL (SEQ ID NO: 6) as determined by an analysis of the DNA from amplified phage clones. A minigene sequence of 5′-GTT,TCT,TGG,TGT,TCT,TCT,TGT,TCT,CTT-3′ (SEQ ID NO: 7) was derived from the analysis and serves as the minigene construct for the development of the Brucella M cell vaccine. The polynucleotide shown above, encoding the selected VSWCSSCSL peptide sequence, was ligated into a plasmid together with immunomodulatory sequences including a CMV promoter for controlling transcription, T cell epitopes (K99) and enhancer sequences. The peptide mimetic for the Brucella LPS epitope was expressed as a trimer to improve immunogenicity. Likewise the K99 fimbrial subunit was expressed as a repetitive trimer on the N-terminus of protein s1. The expression of the K99 fimbrial subunit was included to provide a scaffolding for the LPS minitope and to improve the immunogenicity of the peptide epitope.

### Example 16

Preparation of Minigenes and Minitopes for the Development of Norwalk Virus Vaccines

The Norwalk-like viruses (NLV) are the most important cause of epidemic outbreaks of foodborne and waterborne gastroenteritis. Sequence analyses of viral capsid protein coding regions have identified two major genetic groups of NLV, with many genetic subtypes (and likely antigenic subtypes) within each group (Frankhauser, R., et al., J. Infect. Dis. (1998) 178: 1571-1578.). The NLV are difficult to study because these viruses are refractory to growth in cell culture and small animal models. Thus, the early steps in infection including cell binding and entry have been focused on.

Current studies on the molecular interactions between NLV capsids and cellular receptors were designed to reveal or identify conformational epitope(s) that occur concurrent with the binding event or fusion of viral epitope with the cell receptor. Monoclonal antibodies specific for the fusion-induced conformational epitope are used to select minitopes and prepare minigenes for incorporation into M cell vaccines as described above.

### Example 17

Preparation of Minitopes and Minigenes for the Development of an Anthrax Vaccine

Anthrax produced by the bacterium Bacillus anthracis is an infectious disease resulting from contact with endospores in contaminated animal products or their dusts. Cutaneous anthrax, which accounts for 95% of cases in the world, results from contamination of a lesion in the skin and progresses to fatal septicemia in 10-20% of untreated cases. Inhalation anthrax is nearly always lethal without early, aggressive intervention. The results of a field study with the U.S. military Anthrax Vaccine Adsorbed (AVA) suggested that it prevented cutaneous infection in humans (Demicheli, V. et al., Vaccine (1998) 16: 880-884). Its effectiveness in preventing inhalation disease in humans is questionable in view of failure to protect animals against inhalation anthrax.

The inventors have obtained monoclonal antibodies against two major B. anthracis antigens, PA and poly-γ-D-glutamyl capsular material and protective antigen (PA), a cell surface binding unit of both anthrax edema toxin and lethal toxin. In addition, toxin genes (PA) cloned into E. coli hosts serve as the source of epitope DNA for ligation plasmids and incorporation into M cell vaccine constructs as described above.

### Example 18

The Generation of Mucosal Tolerance using M Cell Delivery of Peptides that Promote Anergy to Self Antigens

Autoimmune diseases such as arthritis (7 different types), multiple sclerosis, uveitis, myasthenia gravis, type 1 diabetes, thyroiditis and colitis respond favorably to the oral delivery of native proteins, sometimes peptides, associated with the tissue under attack by the immune system (Cohen, I., Behring, Inst. Mitt. (1985) 77: 88-94) This phenomenon, referred to as oral tolerance, interrupts and suppresses the autoimmune disease process by stimulating the natural mucosal immune mechanisms in the gut associated lymphoid tissues (Galt) of the small intestine (Hanninen . Scand. J. Immunol. (2000) 52 (3): 217-225;Shi. et al., J. Immunol. (1999) 162 (10): 5757-5763; Haflter., Ann. N.Y Acad. Sci. (1997) 835: 120-131). Mucosal oral tolerance can be induced by three different mechanisms: active suppression, clonal anergy and clonal deletion. Antigen dose is the primary factor determining the form of peripheral tolerance that develops. The generation of tolerance due to regulatory T cells (active suppression) is favored by administration of low doses of antigen, whereas administration of high doses of antigen biases toward development of tolerance due to anergy or deletion.

The distinction between these mechanisms of oral tolerance are not mutually exclusive and they may occur concurrently. In low dose tolerance, regulatory T cells are stimulated to secret suppressive cytokines, such as TGF-b, IL-4 and IL-10 which function to down-regulate the activiated inflammatory Th1 cells. Clonal anergy may result when high doses of oral antigen induce unresponsiveness in the immunoreactive Th1 cell function. The cells are not deleted but are rendered intrinsically incapable of responding to a specific antigen in the context of their T cell receptor and peptide associated with MHC. Anergy may be overcome with high concentrations of IL-2. Clonal deletion results in the elimination of antigen response T cells. In the presence of high concentrations of protein, deletion of cells specific for that protein can occur directly within the Peyer's patch as well as in the thymus. Most current therapeutics for autoimmune disorders are administered orally as native proteins or peptide residues from target tissues or secretion products of cells.

The oral delivery of tissue specific antigens (tolerogens) has generally been accomplished with large or intact proteins which are broken down to fragments by the normal digestive processes (Rosen, A., et al., 1999. Cell. Death Differ. 6: 6-11; Kweon. et al., Digestion 63 suppl S1: 1-11; Lipkowski. Et al. Biofactors (2000) 12: 147-150). Specific fragments or peptides are taken up by antigen-presenting cells (M Cells) and processed for presentation to undifferentiated T cells. These regulatory T cells release cytokines which suppress inflammation (Marth. et al., Gastroenterol. (1999) 37 (2): 165-185: Hafler. et al., Ann. N.Y Acad. Sci (1997) 835: 120-131). An alternate strategy which is contemplated involves the oral delivery of DNA encoding protein fragments or peptides representative of self tissue antigens using the M cell delivery system. The processing of tolerogenic peptide DNA (with the appropriate regulatory T cell epitope or none at all) by M cells, the synthesis of tolerogenic peptides in situ and the subsequent presentation to regulatory T cells in the Peyer's patch will lead to mucosal as well as systemic tolerance. The various self-antigens or tolerogenic peptides that may be represented or presented in DNA coding by way of an M cell directed vaccine as described above include Type II collagen (arthritis) (Weiner. et al., Springer Semin Immunopathol, (1998) 20 (1-2): 289-308), myelin protein MBP, PLP, MOG 9 (multiple sclerosis) (Hafler. et al., Ann. N.Y. Acad. Sci (1997) 835: 120-131), S-Ag, MP (uveitis), ArchR (myasthenia gravis) (Sempowski, G. et al., J. Immunol. (2001) 166: 2808-2817), insulin, GAD (type 1 diabetes) (Bach, J., Endocr. Rev. (1994) 15: 516-523), thyroglobulin (thyroiditis), basement membrane antigen (glomerulonephritis ) (Wilson, C. et al., In The Kidney (1991) Brenner and Rector, eds. W. Saunders, Philadelphia) or colonic proteins (colitis). Such DNA materials may be obtained by PCR with human tissue or, by decoding displayed peptides from phage display using auto-immune antibody directed against the tissue proteins.

### Example 19

Tumor Vaccines: DNA Coding Minitopes of Carbohydrate Tumor Antigens

There are ongoing Phase I/II and III clinical trials with DNA vaccines for treating melanoma, lymphoma, prostate cancer, kidney cancer, and colon cancer. The key to the success of any of these and future DNA vaccines rests with the use of highly immunogenic tumor epitopes in combination with appropriate accessory molecules (cytokines, decoy ligands, targeting molecules) into a carrier system that elicits either a cell mediated or humoral response or both.

The physical characteristics of tumor antigens that would make the most ideal targets for antibody therapeutics or vaccine constructs include cell surface expression; high, stable expression levels in tumor cells; low or absent expression in normal tissues; lack of a soluble form of the antigenic target; and lack of internalization of a antigen/antibody complex. In addition, the type of immune response, either Th1 or Th2-mediated responses, provoked by the epitope is key to the successful eradication of a tumor mass. With the recent progress in molecular biology and gene technology, many new cancer-specific antigens have been identified (UrCarrilho, C. et al., Virchows Arch (2000) 437(2):173-9).

A variety of current and past tumor vaccine formulations are predicated on cell-based technology using either autologous cells (cytokine gene-transduced or hapten-modified, for example) or allogeneic whole cells consisting of single or pooled cell lines. Early efforts to evaluate responses to tumor vaccines have focused on humoral responses. The result was the identification of many antigens that could be defined serologically on tumor cells including glycoproteins, glycolipids, gangliosides and complex proteins. Adoptive therapies directed against these antigens have been disappointing but there is renewed interest in humoral immune responses and in tumor vaccines directed against carbohydrate epitopes.

The dominant thrust of current research in tumor immunobiology has focused on defining antigens recognized by human T cells and on augmenting the cellular immune response to tumors. Consequently, the focus of these efforts have been on protein or oligopeptide tumor antigens. Recent studies have focused on the use vaccines containing oligopeptides or peptides representative of key regions in the tumor cell epitope. ( Qiu, J. et al., Hybridoma (1999) 18: 103-112; Kieber-Emmons, T. et al., J. Immunol. (2000) 165: 623-627).

T cell responsiveness to an epitope is effected both by its affinity for the presenting MHC molecule and the affinity of the MHC-peptide complex for the thymus cell receptor (TCR) (Nagorsen, D., et al., Cancer Res. (2000) 60(17):4850-4). One limitation of cancer immunotherapy is that natural tumor antigens, especially carbohydrate epitopes, elicit relatively weak T cell responses, in part because high-affinity T cells are rendered tolerant to these antigens (Slantsky, J. et al.,: Immunity (2000) 13(4):529-538). Many studies have demonstrated the role of HLA class I-restricted cytotoxic T lymphocytes (CTLs) in cancer specific-immunotherapies (Mond. et al., Ann. Rev. Immunol. (1995) 13: 655-667). The engineering of MHC class I-restricted tumor peptide epitopes that increase the stability of the MHC-peptide-TCR complex are significantly more potent as tumor vaccines (Nagorsen, D., et al., Cancer Res. (2000) 60(17):4850-4). The improved immunity results from enhanced in vivo expansion of T cells specific for the natural tumor epitope. These results indicate that peptides that stabilize the MHC-peptide-TCR complex may provide superior antitumor immunity through enhanced stimulation of specific T cells, and such peptides may be delivered to M cells or coded by DNA minigenes as described above.

### Example 20

Tumor Vaccines: Increasing Immunogenicity with the use of Immunomodulatory Cytokines

Cytokines may be used as immunomodulatory adjuvants to be administered in formulations with the tumor vaccines and other vaccines described herein. For instance, liposomes incorporating interferon gamma have been shown to increase the residence time of the cytokine at the vaccination site as compared to cytokine gene transfection of tumor cells (van Slooten et al., Pharm. Res. Jan.,17, 2000 (1): 42-8). Thus, vaccines may be formulated to include cytokine-containing liposomes admixed with the M cell ligand complexes of the invention. Alternatively, M cell ligand complexes of the present invention may be included together in liposome formulations along with adjuvant cytokines. Also, constructs may be made according to the invention wherein cytokines are otherwise associated with or attached to the M cell ligand and immunogen complexes of the invention.

Liposomes may be made by means that are well known in the art, and may be polymerized or unpolymerized, depending on the desired characteristics for the liposome. In general, polymerized lipid compositions may be produced according to techniques described in U.S. Pat. No. 5,962,422 to Nagy et al., “Inhibition of Selectin Binding”, utilizing the materials and methods disclosed therein. Other references are U.S. Pat. No. 6,342,226 to Betbeder et al., “Method for Increasing Immunogenicity, Product Obtained and Pharmaceutical Compositions”, U.S. Pat. No. 6,090,406 to Popescu et al., “Potentiation of Responses with Liposomal Adjuvants”, and U.S. Pat. No. 6,225,445 to Sen et al., “Methods and Compositions for Lipidiziation of Hydrophilic Molecules.” Such vaccine formulations would contain a ratio of cytokine to vaccine complex that is optimized to produce the desired response.

The foregoing detailed description has been given for clearness of understanding only and no unnecessary limitations should be understood therefrom as modifications will be obvious to those skilled in the art. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. A composition comprising:
an M cell specific ligand;
a nucleic acid sequence encoding an immunogen; and
a nucleic acid binding moiety.

2. The composition of claim 1, wherein said nucleic acid sequence is a DNA sequence.

3. The composition of claim 1, wherein said nucleic acid binding moiety is a polypeptide.

4. The composition of claim 3, wherein said polypeptide comprises a polymeric chain of basic amino acid residues.

5. The composition of claim 4, wherein said polymeric chain comprises polylysine.

6. The composition of claim 3, wherein said polypeptide is a fusion protein further comprising said M cell specific ligand.

7. The composition of claim 1, wherein said immunogen is selected from the group consisting of immunogens expressed by infectious agents and tumor specific antigens.

8. The composition of claim 7, wherein said infectious agent is selected from the group consisting of bacterium, parasite, virus, fungus, prion, tuberculobacillus, leprosy bacillus, malaria parasite, diphtheria bacillus, tetanus bacillus, Leishmania, Salmonella, Schistosoma, measles virus, mumps virus, herpes virus, HIV, cancer and influenza virus.

9. The composition of claim 1, wherein said M cell specific ligand is selected from the group consisting of the protein σ1 of a reovirus, adhesin derived from Salmonella and adhesin derived from polio virus and M cell tropic fragments thereof.

10. The composition of claim 9, wherein said M cell specific ligand is the protein σ1 of a reovirus, a tetramer or trimer thereof, or an M cell tropic fragment thereof.

11. The composition of claim 2, wherein said DNA sequence further comprises a plasmid vector in which said DNA sequence encoding an immunogen is operably linked to transcription regulatory elements.

12. A composition comprising:
an M cell specific ligand
an immunogen and
a linker molecule.

13. The composition of claim 12, wherein said linker is a crosslinker and said M cell specific ligand is conjugated to an immunogen.

14. The composition of claim 13, wherein said crosslinker is selected from the group consisting of SPDP, DSS, STAB, SATA, MBS and GMBS.

15. The composition of claim 12, wherein said linker is a complexing moiety and said M cell ligand is complexed to said immunogen.

16. The composition of claim 15, wherein said complexing moiety is selected from the group consisting of nitrilotriacetic (NTA)-metal complex and iminodiacetic acid (IDA)-metal.

17. A composition comprising:
an M cell specific ligand;
an immunogen; and
a liposome.

18. The composition of claim 17, wherein said immunogen is encapsulated within said liposome and said M cell specific ligand is conjugated to the liposome.

19. The composition of claim 17, wherein said immunogen is surface-displayed on said liposome and said M cell specific ligand is conjugated to the liposome.

20. A composition comprising:
an M cell specific ligand;
an immunogen; and
a polypeptide.

21. The composition of claim 20, wherein said M cell specific ligand and said immunogen are conjugated to said polypeptide.

22. A composition of claim 21, wherein said M cell specific ligand and said immunogen are comprised in a fusion protein or polypeptide.

23. The composition of any of claims 12, 17, 20 and 22, wherein said immunogen is selected from the group consisting of molecules associated with infectious agents and tumor specific antigens.

24. The composition of any of claims 12, 17, 20 and 22, wherein said infectious agent is selected from the group consisting of bacterium, parasite, virus, fungus, prion, tuberculobacillus, leprosy bacillus, malaria parasite, diphtheria bacillus, tetanus bacillus, Leishmania, Salmonella, Schistosoma, measles virus, mumps virus, herpes virus, HIV, cancer and influenza virus.

25. The composition of any of claims 12, 17, 20 and 22, wherein said M cell specific ligand is selected from the group consisting of the protein σ1 of a reovirus, adhesin derived from Salmonella and adhesin derived from polio virus and M cell tropic fragments thereof.

26. The composition of claim 25, wherein said M cell specific ligand is the protein σ1 of a reovirus, a tetramer or trimer thereof, or an M cell tropic fragment thereof.

27. A vaccine comprising the composition of any of claims 1 to 26 and a pharmaceutically acceptable excipient.

28. The vaccine of claim 27, which induces a protective immune response in a vaccinated host against said immunogen.

29. The vaccine of claim 27, further comprising an adjuvant.

30. The vaccine of claim 29, wherein said adjuvant comprises an immunomodulator.

31. The vaccine of claim 30, wherein said immunomodulator is selected from the group consisting of cytokines, lymphokines, interleukins, interferons and growth factors.

32. The vaccine of claim 27, wherein the vaccine is formulated in unit dosage form.

33. The vaccine of claim 27, further packaged with instructions for the use of the vaccine to induce an immune response against said immunogen or against the disease with which said immunogen is associated.

34. The vaccine of claim 27, wherein the vaccine is a therapeutic vaccine.

35. The vaccine of claim 27, wherein the vaccine is formulated for administration through a route selected from the group consisting of oral, nasal, vaginal, rectal and urethral routes of administration.

36. A method for immunizing a host against an immunogen, comprising the step of administering the vaccine of claim 27 to the host.

37. A method for assaying for mucosal immunity comprising the steps of administering the vaccine of claim 27 to an animal which is free of infection of the infectious agent whose antigen is to be tested;
isolating mucosal immune cells from the animal; and
co-incubating said isolated cells with heterologous antigen expressing or presenting cells,
wherein lysing of antigen expressing cells is indicative of mucosal immunity in the animal.

38. The method of claim 37, wherein said mucosal immune cells are isolated from tissues selected from the group consisting of lamina propria tissue, intraepithelial tissue, Peyer's patches, lymph nodes, nasal passages, NALT, adenoids and vaginal epithelium.

39. The method of claim 37, comprising the additional step of evaluating the animal's cytokine profile.

40. An isolated nucleic acid encoding a fusion protein comprising a nucleic acid binding moiety and an M cell specific ligand.

41. The nucleic acid of claim 40, wherein said binding moiety comprises a polymeric chain of basic amino acid residues.

42. The nucleic acid of claim 41, wherein said polymeric chain comprises polylysine.

43. The nucleic acid of claim 40, wherein said M cell ligand is selected from the group consisting of: protein σ1 of a reovirus, adhesin derived from Salmonella and adhesin derived from polio virus and M cell tropic fragments thereof.

44. A vector comprising the nucleic acid of any of claims 40 to 43.

45. The vector of claim 44, wherein said vector is an expression vector.

46. A polypeptide comprising the expression product of the vector of claim 45.

47. The vector of claim 45, wherein said nucleic acid is in operable linkage and wherein the operable linkage is selected from the group consisting of sense and antisense orientations relative to transcriptional elements comprising the vector.

48. A host cell comprising the vector of claim 44.

49. A method of expressing a fusion protein comprising the step of expressing the vector of claim 45.

50. An isolated polypeptide comprising a nucleic acid binding moiety and an M cell specific ligand.

51. The polypeptide of claim 50, wherein said binding moiety comprises a polymeric chain of basic amino acid residues.

52. The polypeptide of claim 51, wherein said polymeric chain comprises polylysine.

53. The polypeptide of claim 50, wherein said M cell ligand is selected from the group consisting of: protein σ1 of a reovirus, adhesin derived from Salmonella and adhesin derived from polio virus and M cell tropic fragments thereof.

54. An isolated antibody that binds to the polypeptide of claim 50.

55. A kit comprising:
an M cell specific ligand; and
a nucleic acid or immunogen binding moiety.

56. The kit of claim 55, wherein said nucleic acid or immunogen binding moiety is a polypeptide.

57. The kit of claim 56, wherein said polypeptide comprises a polymeric chain of basic amino acid residues.

58. The kit of claim 57, wherein said polymeric chain comprises polylysine.

59. The kit of claim 56, wherein said polypeptide is a fusion protein further comprising said M cell specific ligand.

60. The kit of claim 55, wherein said M cell specific ligand is selected from the group consisting of: protein σ1 of a reovirus, adhesin derived from Salmonella and adhesin derived from polio virus and M cell tropic fragments thereof.

61. The kit of claim 60, wherein said M cell specific ligand is the protein σ1 of a reovirus, a tetramer or trimer thereof, or an M cell tropic fragment thereof.

62. The kit of claim 55 further comprising instructions for the use of said M cell specific ligand and said nucleic acid or immunogen binding moiety to deliver a nucleic acid vaccine or other vaccine to mucosal lymphoid tissue.

63. The kit of claim 62 further comprising instructions for measuring a mucosal immune response raised against said nucleic acid vaccine or other vaccine.

64. A pharmaceutical composition formulated for mucosal delivery comprising an M cell specific ligand and an immunogen.


SEQUENCE LISTING (7 sequences)

SEQ ID NO: 1 - DNA, 27 bp, Candida albicans
  CDS (1)..(27): minigene for PS76 immunogen
tatcgtcagtttgtgacgggtttttgg

SEQ ID NO: 2 - protein, 9 aa, Candida albicans
YRQFVTGFW

SEQ ID NO: 3 - protein, 9 aa, Streptococcus sp.
  MISC_FEATURE: Group B streptococcal immunogen
WENWMMGNA

SEQ ID NO: 4 - protein, 12 aa, Streptococcus sp.
  MISC_FEATURE: Group B streptococcal immunogen
FDTGAFDPDWPA

SEQ ID NO: 5 - DNA, 36 bp, Streptococcus sp.
  misc_feature: minigene encoding the immunogen of SEQ ID NO 4
tttgatacgctggcttttgatcctgattggcctgct

SEQ ID NO: 6 - protein, 9 aa, artificial
  immunogen against Brucella A antigen
VSWCSSCSL

SEQ ID NO: 7 - DNA, 27 bp, artificial
  minigene for immunogen against Brucella A antigen
gtttcttggtgttcttcttgttctctt